(19) United States
(12) Patent Application Publication
(10) Pub. No.: US 2009/0209427 A1
(43) Pub. Date: Aug. 20, 2009

(54) Microbial glyphosate resistant epsps

(76) Inventors: Murtaza F. Alibhai, Chesterfield, MO (US); Cathy Chay, Ballwin, MO (US); Stanislaw Flasinski, Chesterfield, MO (US); Maolong Lu, St.Louis, MO (US); William Stallings, Wildwood, MO (US); Douglas Sammons, Wentzville, MO (US)

Correspondence Address: HOWREY LLP, C/O IP DOCKETING DEPARTMENT, 2941 FAIRVIEW PARK DRIVE SUITE 200, FALLS CHURCH, VA, 22042, US

(21) Appl. No.: 11/629,364

(22) PCT Filed: Jun. 20, 2005
(86) PCT No.: PCT/US05/21725

§ 371 Date: Dec. 12, 2006

Related U.S. Application Data
(60) Provisional application No. 60/582,658, filed on Jun. 24, 2004.

(51) Int. Cl. A01N 57/18; C07H 21/04

(52) U.S. Cl. 504/206; 536/23.2

(57) ABSTRACT

The present invention is based, in part, on a method for the identification of glyphosate resistant 5-enolpyruvyl-3-phosphoshikimate synthase polypeptides and the isolation of the DNA molecules that encode the polypeptides. Also, chimeric DNA constructs are described that are useful to transform and express the glyphosate resistant 5-enolpyruvyl-3-phosphoshikimate synthase polypeptide in bacteria and plant cells. The invention provides chimeric DNA molecules that are useful to transform plant cells, and the transformed plants, progeny, and parts thereof regenerated from the transformed plant cells.

### PRIORITY CLAIM

The present application claims priority to U.S. provisional application Ser. No. 60/582,658 filed 24 Jun. 2004, the entire contents of which are hereby incorporated by reference herein.

### FIELD OF THE INVENTION

This invention relates to plant molecular biology and plant genetic engineering. In particular, the invention relates to DNA constructs and methods useful to provide herbicide resistance in plants and, more particularly, to the use of a glyphosate resistant 5-enolpyruvylshikimate-3-phosphate synthase in this method.

### DESCRIPTION OF THE RELATED ART

N-phosphonomethylglycine, also known as glyphosate, is a well-known herbicide that has activity on a broad spectrum of plant species. Glyphosate is the active ingredient of Roundup® (Monsanto Co., St Louis, Mo.), a herbicide having a long history of safe use and a desirably short half-life in the environment. When applied to a plant surface, glyphosate moves systemically through the plant. Glyphosate is phytotoxic due to its inhibition of the shikimic acid pathway, which provides a precursor for the synthesis of aromatic amino acids. Glyphosate inhibits the class I 5-enolpyruvyl-3-phosphoshikimate synthase (EPSPS) found in plants and some bacteria. Glyphosate tolerance in plants can be achieved by the expression of a modified class I EPSPS that has lower affinity for glyphosate, however, still retains their catalytic activity in the presence of glyphosate (U.S. Pat. Nos. 4,535,060, and 6,040,497).

EPSPS enzymes, such as, class II EPSPSs have been isolated from bacteria that are naturally resistant to glyphosate and when the enzyme is expressed as a gene product of a transgene in plants provides glyphosate tolerance to the plants (U.S. Pat. Nos. 5,633,435 and 5,094,945). Enzymes that degrade glyphosate in plant tissues (U.S. Pat. No. 5,463,175) are also capable of conferring plant tolerance to glyphosate. DNA constructs that contain the necessary genetic elements to express the glyphosate resistant enzymes or degradative enzymes create chimeric transgenes useful in plants. Such transgenes are used for the production of transgenic crop plants that are tolerant to glyphosate, thereby allowing glyphosate to be used for effective weed control with minimal concern of crop damage. For example, glyphosate tolerance has been genetically engineered into corn (U.S. Pat. No. 5,554,798), wheat (Zhou et al. Plant Cell Rep. 15:159-163, 1995), soybean (WO 9200377) and canola (WO 9204449).

Development of herbicide-tolerant crops has been a major breakthrough in agriculture biotechnology as it has provided farmers with new weed control methods. One enzyme that has been successfully engineered for resistance to its inhibitor herbicide is class I EPSPS. Variants of class I EPSPS have been isolated (Pro-Ser, U.S. Pat. No. 4,769,061; Gly-Ala, U.S. Pat. No. 4,971,908; Gly-Ala, Gly-Asp, U.S. Pat. No. 5,310,667; Gly-Ala, Ala-Thr, U.S. Pat. No. 5,8866,775, Thr-Ile, Pro-Ser, U.S. Pat. No. 6,040,497) that are resistant to glyphosate. Although, many EPSPS variants either do not demonstrate a sufficiently high K; for glyphosate or have a Kₘ for phosphoenol pyruvate (PEP) too high to be effective as a glyphosate resistance enzyme for use in plants (Padgette et. al, In “Herbicide-resistant Crops”, Chapter 4 pp 53-83. ed. Stephen Duke, Lewis Pub, CRC Press Boca Raton, Fla. 1996).

There is a need in the field of plant molecular biology for a diversity of genes that can provide a positive selectable marker phenotype and an agronomically useful phenotype. In particular, glyphosate tolerance is used extensively as a positive selectable marker in plants and is a valuable phenotype for use in crop production. The stacking and combining of existing transgene traits with newly developed traits is enhanced when distinct positive selectable marker genes are used. The marker genes provide either a distinct phenotype, such as, antibiotic or herbicide tolerance, or a molecular distinction discernable by methods used for protein and DNA detection. The transgenic plants can be screened for the stacked traits by analysis for multiple antibiotic or herbicide tolerance or for the presence of novel DNA molecules by DNA detection methods.

The present invention provides chimeric genes for the expression of glyphosate resistant EPSPS enzymes. These enzymes and the DNA molecules that encode them are useful for the genetic engineering of plant tolerance to glyphosate herbicide.

### BRIEF DESCRIPTION OF THE DRAWINGS

FIG. 1. Plasmid map illustrating pMON58454

FIG. 2. Plasmid map illustrating pMON42488

FIG. 3. Plasmid map illustrating pMON58477

FIG. 4. Plasmid map illustrating pMON76553

FIG. 5. Plasmid map illustrating pMON58453

FIG. 6. Plasmid map illustrating pMON21104

FIG. 7. Plasmid map illustrating pMON70461

FIG. 8. Plasmid map illustrating pMON81523

FIG. 9. Plasmid map illustrating pMON81524

FIG. 10. Plasmid map illustrating pMON81517

FIG. 11. Plasmid map illustrating pMON58481

FIG. 12 Plasmid map illustrating pMON81546

FIG. 13 Plasmid map illustrating pMON68922

FIG. 14. Plasmid map illustrating pMON68921

FIG. 15. Plasmid map illustrating pMON58469

FIG. 16. Plasmid map illustrating pMON81568

FIG. 17. Plasmid map illustrating pMON81575

### SUMMARY OF THE INVENTION

A chimeric DNA molecule comprising a polynucleotide molecule encoding a glyphosate resistant EPSPS enzyme, wherein said EPSPS enzyme comprises the sequence domains X₁-D-K-S (SEQ ID NO:1), in which X₁ is G or A or S or P; S-A-Q-X₂-K (SEQ ID NO:2), in which X₂ is any amino acid; and R-X₃-X₄-X₅-X₆ (SEQ ID NO:3), in which X₃ is D or N, X₄ is Y or H, X₅ is T or S, X₆ is R or E; and N-X₇-X₈-R (SEQ ID NO:4), in which X₇ is P or E or Q, and X₈ is R or L. Additionally, a chimeric DNA molecule comprising a promoter molecule functional in a plant cell further comprises a DNA molecule encoding a chloroplast transit peptide operably linked to the DNA molecule that encodes a glyphosate resistant EPSPS enzyme of the present invention to direct the EPSPS enzyme into a chloroplast of the plant cell. Exemplary EPSPS enzyme polypeptide sequences of the present invention are disclosed in SEQ ID NOs: 5-18.

In another aspect of the invention, a chimeric DNA molecule is provided that comprises a polynucleotide molecule coding sequence for a glyphosate resistant EPSPS enzyme of the present invention, wherein the polynucleotide molecule is selected from the group consisting of SEQ ID NO: 19-32. In yet another aspect of the invention, a chimeric DNA molecule is provided that comprises a polynucleotide molecule coding sequence for a glyphosate resistant EPSPS enzyme of the present invention, wherein the polynucleotide molecule has been modified for enhanced expression in plant cells. The modified polynucleotide molecule is an artificial DNA molecule that encodes an EPSPS enzyme substantially identical to SEQ ID NO: 5-18, the artificial DNA molecule is an aspect of the present invention. Exemplary artificial DNA molecules are disclosed in SEQ ID NO: 33-37.

In yet another aspect of the invention is a plant cell transformed with a chimeric DNA molecule of the present invention. The chimeric DNA comprising a polynucleotide selected from the group consisting of SEQ ID NO: 5-18 and 33-37. The plant cell can be a monocot or a dicot plant-cell. The plant cell is regenerated into an intact transgenic plant. The transgenic plant and progeny thereof are treated with glyphosate and selected for tolerance to glyphosate. Furthermore, a transgenic plant and progeny thereof comprising the chimeric DNA molecule is an aspect of the present invention. Additionally, a transgenic plant and progeny thereof expressing in its cells and tissues the EPSPS enzymes of the present invention is an aspect of the invention.

The invention provides a method is provided for selectively killing weeds in a field of crop plants comprising the steps of: a) planting crop seeds or plants that are glyphosate tolerant as a result of a chimeric DNA molecule being inserted into said crop seeds or plants, said chimeric DNA molecule comprising (i) a promoter region functional in a plant cell; and (ii) a DNA molecule that encodes a glyphosate resistant EPSPS of the present invention; and (iii) a transcription termination region; and b) applying to said crop seeds or plants a sufficient amount of glyphosate that inhibits the growth of glyphosate sensitive plants, wherein said amount of glyphosate does not significantly affect said crop seeds or plants that comprise the chimeric gene.

In another aspect of the invention a method is provided for identifying a glyphosate resistant EPSPS enzyme comprising identifying a S-A-Q-X-K amino acid motif in the EPSPS enzyme, where X is any amino acid. An isolated glyphosate resistant EPSPS enzyme comprising a S-A-Q-X-K amino acid motif in the EPSPS enzyme, where X is any amino acid, and the motifs -G-D-K-X₃- in which X₃ is Ser or Thr, and R-X₁-H-X₂-E- in which X₁ is an uncharged polar or acidic amino acid and X₂ is Ser or Thr, and -N-X₅-T-R- in which X₅ is any amino acid are not present. A transgenic plant and progeny thereof comprising a chimeric DNA molecule comprising an isolated glyphosate resistant EPSPS enzyme comprising a S-A-Q-X-K amino acid motif in the EPSPS enzyme, where X is any amino acid, and the motifs -G-D-K-X₃- in which X₃ is Ser or Thr, and R-X₁-H-X₂-E- in which X₁ is an uncharged polar or acidic amino acid and X₂ is Ser or Thr, and -N-X₅-T-R- in which X₅ is any amino acid are not present.

A method is also provided for producing a glyphosate tolerant plant comprising the steps of: a) transforming a plant cell with the chimeric DNA molecule of the present invention; and b) regenerating said plant cell into an intact plant; and c) selecting said plant for tolerance to glyphosate.

The present invention provides for a method for identifying a transgenic glyphosate tolerant plant seed comprising the steps of: a) isolating genomic DNA from said seed; and b) hybridizing a DNA primer molecule to said genomic DNA, wherein said DNA primer molecule is homologous or complementary to a portion of the DNA sequence selected from the group consisting of SEQ ID NO: 19-32, and 33-37; and c) detecting said hybridization product.

In another aspect of the invention is a DNA molecule comprising a wheat GBSS (Granule bound starch synthase, GBSS) chloroplast transit peptide (CTP) coding sequence encoding a polypeptide substantially identical to SEQ ID NO: 38 operably connected to a glyphosate resistant. EPSPS coding sequence. Exemplary fusion polypeptides of the wheat GBSS CTP, (TS-Ta.Wxy) and glyphosate resistant EPSPS include, but are not limited to SEQ ID NO: 39, SEQ ID NO: 40 and SEQ ID NO: 41. A transformed plant and progeny thereof comprising SEQ ID NO: 39, SEQ ID NO: 40 or SEQ ID NO: 41 is an aspect of the invention. The present invention further contemplates the use of a wheat GBSS CTP operably linked to a heterologous protein for transport into a plant chloroplast, wherein the heterologous protein provides an agronomically useful phenotype to the plant.

### DETAILED DESCRIPTION OF THE INVENTION

The following descriptions are provided to better define the present invention and to guide those of ordinary skill in the art in the practice of the present invention.

The present invention describes polynucleotide and polypeptide molecules of glyphosate resistant, EPSPS enzymes. Chimeric DNA molecules were designed to produce the EPSPS enzymes in transgenic cells and provide for analysis of the EPSPS enzyme activity and glyphosate resistance. Chimeric DNA molecules mean any DNA molecule comprising heterologous regulatory and coding sequences that are not found together in nature. Accordingly, a chimeric DNA molecule may comprise regulatory sequences and coding sequences that are derived from different sources, or regulatory sequences and coding sequences derived from the same source, but arranged in a manner different than that found in nature. In one aspect of the invention, the chimeric DNA molecules were designed to produce the glyphosate resistant EPSPS enzymes in transgenic plant cells in sufficient amount to provide glyphosate tolerance to the plant cells. A transgenic plant cell contains the chimeric DNA molecule in its genome by a transformation procedure resulting in a transgenic plant. The term “genome” as it applies to plant cells encompasses not only chromosomal DNA found within the nucleus, but organelle DNA found within subcellular components of the cell. The term “plant” encompasses any higher plant and progeny thereof, including monocots (e.g., corn, rice, wheat, barley, etc.), dicots (e.g., soybean, cotton, canola, tomato, potato, Arabidopsis, tobacco, etc.), gymnosperms (pines, firs, cedars, etc.) and includes parts of plants, including reproductive units of a plant (e.g., seeds, bulbs, tubers, fruit, flowers, etc.) or other parts or tissues from that which the plant can be reproduced. The term “germplasm” refers to the reproducible living material that contains within it genetic information such as DNA, for example, the living material maybe cells, seeds, pollen, ovules, or vegetative propagules such as tuber and rhizomes. Transgenic germplasm contains the chimeric DNA molecules of the present invention and the additional genetic information naturally contained within the germplasm. The value of the germplasm can be substantially enhanced with the addition of a transgene.

Grain is often produced from transgenic crop plants that contain the chimeric DNA molecules described in the present invention. The grain can be used as food or animal feed and can be further processed to provide useful materials, for example, fiber, protein, oil, and starch. One aspect of the present invention is a material processed from the grain that contains the chimeric DNA molecule of the present invention. Vegetative tissues can also be processed into feed or food products, the DNA molecules of the present invention can be detected and isolated if necessary from the materials processed from the transgenic germplasm. The DNA molecules are useful as markers to track the product in the food system.

Polynucleic acids of the present invention introduced into the genome of a plant cell can therefore be either chromosomally-integrated or organelle-localized. The EPSPS of the present invention can be targeted to the chloroplast by a heterologous chloroplast transit peptide (CTP) fused to the N-terminus of the EPSPS polypeptide creating a chimeric polypeptide molecule. Alternatively, the gene encoding the EPSPS may be integrated into the chloroplast genome, thereby eliminating the need for a chloroplast transit peptide (U.S. Pat. Nos. 6,271,444 and 6,492,578).

In general, the transgenic plant cells are regenerated into intact transgenic plants and the plants are assayed for tolerance to glyphosate herbicide. “Tolerant” or “tolerance” refers to a reduced effect of an agent on the growth and development, and yield of a plant and in particular tolerance to the phytotoxic effects of glyphosate herbicide. Provided herein is the construction of these chimeric DNA molecules, analysis of glyphosate resistance of the EPSPS enzymes, and analysis of plants containing the DNA molecules for tolerance to glyphosate.

“Glyphosate” refers to N-phosphonomethylglycine and its' salts, Glyphosate is the active ingredient of Roundup®E herbicide (Monsanto Co.). Plant treatments with “glyphosate” refer to treatments with the Roundup® or Roundup Ultra® herbicide formulation, unless otherwise stated. Glyphosate as N-phosphonomethylglycine and its' salts (not formulated Roundup® herbicide) are components of synthetic culture media used for the selection of bacteria and plant tolerance to glyphosate or used to determine enzyme resistance in in vitro biochemical assays. Examples of commercial formulations of glyphosate include, without restriction, those sold by Monsanto Company as ROUNDUP®, ROUNDUP® ULTRA, ROUNDUP® ULTRAMAX, ROUNDUP® WEATHERMAX, ROUNDUP® CT, ROUNDUP® EXTRA, ROUNDUP® BIACTIVE, ROUNDUP® BIOFORCE, RODEO®, POLARIS®, SPARK® and ACCOR® herbicides, all of which contain glyphosate as its isopropylammonium salt; those sold by Monsanto Company as ROUNDUP® DRY and RIVAL® herbicides, which contain glyphosate as its ammonium salt; that sold by Monsanto Company as ROUNDUP® GEOFORCE, which contains glyphosate as its sodium salt; and that sold by Zeneca Limited as TOUCHDOWN® herbicide, which contains glyphosate as its trimethylsulfonium salt. Glyphosate herbicide formulations can be safely used over the top of glyphosate tolerant crops to control weeds in a field at rates as low as 8 ounces/acre upto 64 ounces/acre. Experimentally, glyphosate has been applied to glyphosate tolerant crops at rates as low as 4 ounces/acre and upto or exceeding 128 ounces/acre with no substantial damage to the crop plant.

EPSPS enzymes have been isolated that are naturally resistant to inhibition by glyphosate, these have been identified as class II EPSPS enzymes (U.S. Pat. No. 5,633,435). The class II enzymes are different from other EPSPS enzymes by containing four distinct peptide motifs. These motifs were identified in U.S. Pat. No. 5,633,435 as -G-D-K-X₃- in which X₃ is Ser or Thr, and -S-A-Q-X₄-K- in which X₄ is any amino acid, and R-X₁-H-X₂-E- in which X₁ is an uncharged polar or acidic amino acid and X₂ is Ser or Thr, and -N-X₅-T-R- in which X₅ is any amino acid.

The present invention identifies a new class of glyphosate resistant EPSPS enzymes, for which a chimeric DNA molecule comprising a polynucleotide encoding the glyphosate resistant EPSPS comprises the sequence domains of motif #1 X₁-D-K-S (SEQ ID NO: 1), in which X₁ is G or A or S or P; motif #2 S-A-Q-X₂-K (SEQ ID NO:2), in which X₂ is any amino acid; and motif #3 R-X₃-X₄-X₅-X₆ (SEQ ID NO:3), in which X₃ is D or N, X₄ is Y or H, X₅ is T or S, X₆ is R or E; and motif #4 N-X₇-X₈-R (SEQ ID NO:4), in which X₇ is P or E or Q; and X₈ is R or L is an aspect of the present invention. The chimeric DNA molecule may further comprise additional coding polynucleic acid sequences, for example those encoding additional proteins such as a chloroplast transit peptide in the same coding translational reading frame as the EPSPS coding sequence, and noncoding polynucleic acid sequences, such as, promoter molecules, introns, leaders, and 3′ termination regions.

A method useful for identifying a glyphosate resistant EPSPS enzyme has been developed in which the S-A-Q-X-K motif is identified in the EPSPS protein, where X is any amino acid. Bioinformatic analysis of protein sequence collections, for example, those contained in Genbank (NIH genetic sequence database) or other data collections found in the NCBI (National Center for Biotechnology Information) can identify glyphosate resistant EPSPS enzymes containing the SAQXK motif. The EPSPS enzymes of the new EPSPS class of the present invention have additional peptide motifs identified as distinct from those defining class II EPSPS enzymes as shown in Table 1. Further analysis of four motifs of EPSPS subdivides the new classification of glyphosate resistant EPSPS into three subclasses. The first subclass is represented by the EPSPS polypeptide and polynucleotide sequences from Xylella fastidiosa (XYL202310, SEQ ID NO: 5 and SEQ ID NO: 19, respectively) and Xanthoinonas campestris (XAN202351, SEQ ID NO: 6 and SEQ ID NO: 20, respectively). The motifs that define the first subclass are GDKS; SAQX₁K₁ where X₁ is I or V; RDYTR; and NPRR. The second subclass is represented by the EPSPS polypeptide and polynucleotide sequences isolated from Rhodopseudomonas palustris (RHO102346, SEQ ID NO: 7 and SEQ ID NO: 21, respectively), Magnetospirillum magnetotacticum (Mag306428, SEQ ID NO: 8 and SEQ ID NO: 22), and Caulobacter crescentus (Cau203563, SEQ ID NO: 9 and SEQ ID NO: 23, respectively). The motifs that define the second subclass are GDKS; SAQX₁K₁ where X₁ is I or V; RDHTR; NX₂LR, where X₂ is P or E. The third subclass is represented by EPSPS polypeptide and polynucleotide sequences isolated from Magnetococcus MC-1 (Mag200715, SEQ ID NO: 10 and SEQ ID NO: 24, respectively), Enterococcus faecalis (ENT219801, SEQ ID NO: 11 and SEQ ID NO: 25, respectively), Enterococcus faecalis (EFA101510, SEQ ID NO: 12 and SEQ ID NO: 26, respectively), Enterococcus faecium (EFM101480, SEQ ID NO: 13 and SEQ ID NO: 27, respectively), Thermotoga maritima (TM0345, SEQ ID NO: 14 and SEQ ID NO: 28, respectively), Aquifex aeolicus (AAE101069, SEQ ID NO: 15 and SEQ ID NO: 29, respectively), Helicobacter pylori (HPY200976, SEQ ID NO: 16 and SEQ ID NO: 30, respectively), Helicobacter pylori (BP0401, SEQ ID NO: 17 and SEQ ID NO: 31, respectively), Campylobacter jejuni (CJU10895, SEQ ID NO: 18 and SEQ ID NO: 32, respectively). The motifs that define the third subclass are X₁DXS, where X₁ is A or S or P; SAQVK; RX₂HTE, where X₂ is D or N; NX₃TR, where X₃ is Q or P.

TABLE 1
EPSPS polypeptide motifs

| SEQ ID | | | | | |
|---|---|---|---|---|---|
| NO: | EPSPS | Motif1 | Motif2 | Motif3 | Motif4 |
| 5, 19 | XYL202310 | GDKS | SAQIK | RDYTR | NPRR |
| 6, 20 | XAN202351 | GDKS | SAQVK | RDYTR | NPRR |
| 7, 21 | RHO102346 | GDKS | SAQIK | RDHTE | NPLR |
| 8, 22 | Mag306428 | GDKS | SAQVK | RDHTE | NPLR |
| 9, 23 | Cau203563 | GDKS | SAQVK | RDHTE | NELR |
| 10, 24 | Mag200715 | ADKS | SAQVK | RDHTE | NPTR |
| 11, 25 | ENT219801 | SDKS | SAQVK | RDHTE | NQTR |
| 12, 26 | EFA101510 | SDKS | SAQVK | RDHTE | NQTR |
| 13, 27 | EFM101480 | ADKS | SAQVK | RNHTE | NPTR |
| 14, 28 | TM0345 | PDKS | SAQVK | RDHTE | NPTR |
| 15, 29 | AAE101069 | SDKS | SAQVK | RDHTE | NPTR |
| 16, 30 | HPY200976 | SDKS | SAQVK | RNHTE | NPTR |
| 17, 31 | HP0401 | SDKS | SAQVK | RNHTE | NPTR |
| 18, 32 | CJU10895 | ADKS | SAQVK | RNHSE | NPTR |
| | Class II EPSPS | GDKX1₁ | SAQX₂K | RX₃HX₄K | NX₅TR |

The DNA coding sequence representative of each EPSPS subclass is isolated from genomic DNA extracted from the source organism. The native gene encoding the EPSPS from bacterial source organisms may be referred to herein as the aroA gene or EPSPS coding sequence. The method of isolation involves the use of DNA primer molecules homologous or complementary to the target DNA molecule. The target DNA molecule is isolated from the genomic DNA by a DNA amplification method known as polymerase chain reaction (PCR). This method uses an enzymatic technique to create multiple copies of one sequence of the target polynucleic acid, in the present invention the target DNA molecule encodes the glyphosate resistant EPSPS enzyme. The basis of this amplification method is multiple cycles of temperature changes to denature, then re-anneal the DNA primer molecules, followed by extension to synthesize new DNA strands in the region located between the flanking DNA primers. In general, DNA amplification can be accomplished by any of the various polynucleic acid amplification methods known in the art, including PCR. A variety of amplification methods are known in the art and are described, inter alia, in U.S. Pat. Nos. 4,683,195 and 4,683,202 and in PCR Protocols: A Guide to Methods and Applications, ed. Innis et al., Academic Press, San Diego, 1990. PCR amplification methods have been developed to amplify up to 22 kb (kilobase) of genomic DNA and up to 42 kb of bacteriophage DNA (Cheng et al., Proc. Natl. Acad. Sci. USA 91:5695-5699, 1994). These methods, as well as other methods known in the art of DNA amplification may be used in the practice of the present invention.

The nucleic acid probes and primers of the present invention hybridize under stringent conditions to a target DNA sequence. Hybridization refers to the ability of a strand of nucleic acid to join with a complementary strand via base pairing. Hybridization occurs when complementary sequences in the two nucleic acid strands bind to one another. Nucleic acid molecules or fragments thereof are capable of specifically hybridizing to other nucleic acid molecules under certain circumstances. As used herein, two nucleic acid molecules are said to be capable of specifically hybridizing to one another if the two molecules are capable of forming an anti-parallel, double-stranded nucleic acid structure. A nucleic acid molecule is said to be the “complement” of another nucleic acid molecule if they exhibit complete complementarity. As used herein, molecules are said to exhibit “complete complementarity” when every nucleotide of one of the molecules is complementary to a nucleotide of the other. Two molecules are said to be “minimally complementary” if they can hybridize to one another with sufficient stability to permit them to remain annealed to one another under at least conventional “low-stringency” conditions. Similarly, the molecules are said to be “complementary” if they can hybridize to one another with sufficient stability to permit them to remain annealed to one another under conventional “high-stringency” conditions. Conventional stringency conditions are described by Sambrook et al., 1989, and by Haymes et al., In: Nucleic Acid Hybridization, A Practical Approach, IRL Press, Washington, D.C. (1985), hence forth referred to as Sambrook et al., 1989. Departures from complete complementarity are therefore permissible, as long as such departures do not completely preclude the capacity of the molecules to form a double-stranded structure. In order for a nucleic acid molecule to serve as a primer or probe it need only be sufficiently complementary in sequence to be able to form a stable double-stranded structure under the particular solvent and salt concentrations employed.

As used herein, a substantially homologous DNA molecule is a polynucleic acid molecule that will specifically hybridize to the complement of the polynucleic acid to which it is being compared under high stringency conditions. The term “stringent conditions” is functionally defined with regard to the hybridization of a nucleic-acid probe to a target nucleic acid (i.e., to a particular nucleic-acid sequence of interest) by the specific hybridization procedure discussed in Sambrook et al., 1989, at 9.52-9.55. See also, Sambrook et al., 1989 at 9.47-9.52, 9.56-9.58; Kanehisa, (Nucl. Acids Res. 12:203-213, 1984); and Wetmur and Davidson, (J. Mol. Biol. 31:349-370, 1988). Accordingly, the nucleotide-sequences of the invention may be used for their ability to selectively form duplex molecules with complementary stretches of DNA fragments. Depending on the application envisioned, one can employ varying conditions of hybridization to achieve varying degrees of selectivity of probe towards target sequence. For applications requiring high selectivity, one will typically desire to employ relatively high stringent conditions to form the hybrids, e.g., one will select relatively low salt and/or high temperature conditions, such as provided by about 0.02 M to about 0.15 M NaCl at temperatures of about 50° C. to about 70° C. A high stringent condition, for example, is to wash the hybridization filter at least twice with high-stringency wash buffer (0.2×SSC, 0.1% SDS, 65° C.). Appropriate moderate stringency conditions that promote DNA hybridization, for example, 6.0× sodium chloride/sodium citrate (SSC) at about 45° C., followed by a wash of 2.0×SSC at 50° C., are known to those skilled in the art or can be found in Current Protocols in Molecular Biology, John Wiley & Sons, N.Y. (1989), 6.3.1-6.3.6. Additionally, the salt concentration in the wash step can be selected from a low stringency of about 2.0×SSC at 50° C. to a high stringency of about 0.2×SSC at 50° C. Additionally, the temperature in the wash step can be increased from low stringency conditions at room temperature, about 22° C., to high stringency conditions at about 65° C. Both temperature and salt may be varied, or either the temperature or the salt concentration may be held constant while the other variable is changed. Such selective conditions tolerate little mismatch between the probe and the template or target strand. Detection of DNA sequences via hybridization is well known to those of skill in the art, and the teachings of U.S. Pat. Nos. 4,965,188 and 5,176,995 are exemplary of the methods of hybridization analyses. The present invention provides for a method for identifying a transgenic glyphosate tolerant plant seed comprising the steps of: a) isolating genomic DNA from the seed; and b) hybridizing a DNA probe or primer molecule to the genomic DNA, wherein the DNA probe or primer molecule is homologous or complementary to a portion of the DNA sequence selected from the group consisting of SEQ ID NO: 19-32, and 33-37; and c) detecting the hybridization product. The method can be deployed in DNA detection kits that are developed using the compositions disclosed herein and the methods well known in the art of DNA detection.

The EPSPS coding polynucleotide molecule of the present invention is defined by a nucleotide sequence, which as used herein means the linear arrangement of nucleotides to form a polynucleotide of the sense and complementary strands of a polynucleic acid molecule either as individual single strands or in the duplex. As used herein both terms “a coding sequence” and “a coding polynucleotide molecule” mean a polynucleotide molecule that is translated into a polypeptide, usually via mRNA, when placed under the control of appropriate regulatory molecules. The boundaries of the coding sequence are determined by a translation start codon at the 5′-terminus and a translation stop codon at the 3′-terminus. A coding sequence can include, but is not limited to, genomic DNA, cDNA, and chimeric polynucleotide molecules. A coding sequence can be an artificial DNA. An artificial DNA, as used herein means a DNA polynucleotide molecule that is non-naturally occurring. Artificial DNA molecules can be designed by a variety of methods, such as, methods known in the art that are based upon substituting the codon(s) of a first polynucleotide to create an equivalent, or even an improved, second-generation artificial polynucleotide, where this new artificial polynucleotide is useful for enhanced expression in transgenic plants. The design aspect often employs a codon usage table, the table is produced by compiling the frequency of occurrence of codons in a collection of coding sequences isolated from a plant, plant type, family or genus. Other design aspects include reducing the occurrence of polyadenylation signals, intron splice sites, or long AT or GC stretches of sequence (U.S. Pat. No. 5,500,365). Full length coding sequences or fragments thereof can be made of artificial DNA using methods known to those skilled in the art.

In particular embodiments of the present invention, an artificial DNA encodes polypeptides of a glyphosate resistant EPSPS, for example, artificial DNA molecules of the present invention are constructed using various codon usage tables and methods described in WO04009761, such as, Tm.aroA.nno-Gm (SEQ ID NO: 33), Cc.aroA.nno-At (SEQ ID NO: 34), Xc.aroA.nno-At (SEQ ID NO: 35), Cc.aroA.nno-mono (SEQ ID NO: 36), Xc.aroA.nno-mono (SEQ ID NO: 37), that are contemplated to be useful for at least one of the following: to confer glyphosate tolerance in a transformed plant cell or transgenic plant, to improve expression of the glyphosate resistant enzyme in plants, and for use as selectable markers for introduction of other traits of interest into a plant.

The polynucleic acid molecules encoding the glyphosate resistant EPSPS polypeptides of the present invention may be combined with other non-native, or “heterologous” polynucleotide sequences in a variety of ways. By “heterologous” sequences it is meant any sequence that is not naturally found joined to the poly-nucleotide sequence encoding a polypeptide of the present invention. Of particular interest are various genetic regulatory molecules joined to provide expression of the EPSPS polypeptides in bacteria or plant cells.

Heterologous genetic regulatory molecules are components of the polynucleic acid molecules of the present invention, and when operably linked provide a transgene that include polynucleotide molecules located upstream (5′ non-coding sequences), within, or downstream (3′ non-translated sequences) of a polynucleotide sequence, and that influence the transcription, RNA processing or stability, or translation of the associated polynucleotide sequence. Regulatory molecules may include, but are not limited to promoters, translation leaders (e.g., U.S. Pat. No. 5,659,122), introns (e.g., U.S. Pat. No. 5,424,412), and transcriptional termination regions.

The chimeric DNA molecule of the present invention can, in one embodiment, contain a promoter that causes the overexpression of an EPSPS polypeptide, where “overexpression” means the expression of a polypeptide either not normally present in the host cell, or present in said host cell at a higher level than that normally expressed from the endogenous gene encoding the polypeptide. Promoters, which can cause the overexpression of the polypeptide of the present invention, are generally known in the art, for example, plant viral promoters (P-CaMV35S, U.S. Pat. No. 5,352,605; P-FMV35S, U.S. Pat. Nos. 5,378,619 and 5,018,100), and various plant derived promoters, for example, plant actin promoters (P-Os.Act1, U.S. Pat. Nos. 5,641,876 and 6,429,357), or chimeric combinations of both (for example U.S. Pat. No. 6,660,911).

The expression level or pattern of the promoter of the DNA construct of the present invention may be modified to enhance its expression. Methods known to those of skill in the art can be used to insert enhancing elements (for example, subdomains of the CaMV35S promoter, Benfey et al., EMBO J. 9: 1677-1684, 1990) into the 5′ sequence of genes. In one embodiment, enhancing elements may be added to create a promoter, which encompasses the temporal and spatial expression of the native promoter of the gene of the present invention, but have quantitatively higher levels of expression. Similarly, tissue specific expression of the promoter can be accomplished through modifications of the 5′ region of the promoter with elements determined to specifically activate or repress gene expression (for example, pollen specific elements, Eyal et al., 1995 Plant Cell 7: 373-384). The term “promoter sequence” or “promoter” means a polynucleotide molecule that is capable of, when located in cis to a structural polynucleotide sequence encoding a polypeptide, functions in a way that directs expression of one or more mRNA molecules that encodes the polypeptide. Such promoter regions are typically found upstream of the trinucleotide, ATG, at the start site of a polypeptide coding region. Promoter molecules can also include DNA sequences from which transcription of noncoding RNA molecules occurs, such as antisense RNA, transfer RNA (tRNA) or ribosomal RNA (rRNA) sequences are initiated. Transcription involves the synthesis of a RNA chain representing one strand of a DNA duplex. The sequence of DNA required for the transcription termination reaction is called the 3′ transcription termination region.

It is preferred that the particular promoter selected should be capable of causing sufficient expression to result in the production of an effective amount of an EPSPS enzyme of the present invention to enable glyphosate tolerance to a plant cell. In addition to promoters that are known to cause transcription of DNA in plant cells, other promoters may be identified for use in the current invention by screening a plant cDNA library for genes that are selectively or preferably expressed in the target tissues and then determine the promoter regions from genomic DNA libraries.

It is recognized that additional promoters that may be utilized in the present invention are described, for example, in U.S. Pat. Nos. 6,660,911; 5,378,619; 5,391,725; 5,428,147; 5,447,858; 5,608,144; 5,608,144; 5,614,399; 5,633,441; 5,633,435; and 4,633,436. It is further recognized that the exact boundaries of regulatory sequences may not be completely defined and that DNA fragments of different lengths may have identical promoter activity. Those of skill in the art can identify promoters in addition those herein described that function in the present invention to provide expression of the glyphosate tolerant EPSPS enzyme in a plant cell.

The translation leader sequence is a DNA genetic element means located between the promoter sequence of a gene and the coding sequence. The translation leader sequence is present in the fully processed mRNA upstream of the translation start sequence. The translation leader sequence may affect processing of the primary transcript to mRNA, mRNA stability or translation efficiency. Examples of translation leader sequences include maize and petunia heat shock protein leaders (U.S. Pat. No. 5,362,865), plant virus coat protein leaders, plant rubisco leaders, among others (Turner and Foster, Molecular Biotechnology 3:225, 1995).

Transit peptides generally refer to peptide molecules that when linked to a protein of interest directs the protein to a particular tissue, cell, subcellular location, or cell organelle. Examples include, but are not limited to, chloroplast transit peptides, nuclear targeting signals, and vacuolar signals. The chloroplast transit peptide is of particular utility in the present invention to direct expression of the EPSPS enzyme to the chloroplast. A chloroplast transit peptide (CTP), also referred to as a transit signal (TS-) can be engineered to be fused to the N terminus of proteins that are to be targeted into the plant chloroplast. Many chloroplast-localized proteins are expressed from nuclear genes as precursors and are targeted to the chloroplast by a CTP that if removed during the import steps. Examples of chloroplast proteins include the small subunit (RbcS2) of ribulose-1,5,-bisphosphate carboxylase, ferredoxin, ferredoxin oxidoreductase, the light-harvesting complex protein I and protein II, and thioredoxin F. It has been demonstrated in vivo and in vitro that non-chloroplast proteins may be targeted to the chloroplast by use of protein fusions with a CTP and that a CTP is sufficient to target a protein to the chloroplast. Incorporation of a suitable chloroplast transit peptide, such as, the Arabidopsis thaliana EPSPS CTP (Klee et al., Mol. Gen. Genet. 210:437-442, 1987), and the Petunia hybrida EPSPS CTP (della-Cioppa et al., Proc. Natl. Acad. Sci. USA 83:6873-6877, 1986) has been shown to target heterologous protein to chloroplasts in transgenic plants. The wheat GBSS (Granule bound starch synthase) CTP (TS-Ta.Wxy, SEQ ID NO: 38) of the present invention has shown to provide unexpected high precision in processing at the desirable amino acid site. For example, the polypeptide molecules where wheat GBSS CTP fused is with CP4 EPSPS (SEQ ID NO: 39), or Xc EPSPS (SEQ ID NO: 40), or Cc EPSPS (SEQ ID NO: 41) is an aspect of the present invention. Those skilled in the art will recognize that various chimeric constructs can be made that utilize the functionality of a particular CTP to import a heterologous EPSPS into the plant cell chloroplast. Additionally, the isolated wheat GBSS CTP can be operably linked to heterologous coding sequences of agronomic importance to provide transport of the polypeptide to the plant chloroplast and result in a high precision of transit peptide processing. Agronomically important proteins that benefit from import into chloroplasts are those that are unstable in the plant cytoplasm or are toxic to the plant cell when present in the cytoplasm.

The 3′ non-translated sequence or 3′ transcription termination region means a DNA molecule linked to and located downstream of a structural polynucleotide molecule and includes polynucleotides that provide polyadenylation signal and other regulatory signals capable of affecting transcription, mRNA processing or gene expression. The polyadenylation signal functions in plants to cause the addition of polyadenylate nucleotides to the 3′ end of the mRNA precursor. The polyadenylation sequence can be derived from the natural gene, from a variety of plant genes, or from T-DNA genes. An example of a 3′ transcription termination region is the nopaline synthase 3′ region (nos 3′; Fraley et al., Proc. Natl. Acad. Sci. USA 80: 4803-4807, 1983). The use of different 3′ nontranslated regions is exemplified by Ingelbrecht et al., (Plant Cell 1:671-680, 1989).

The laboratory procedures in recombinant DNA technology used herein are those well known and commonly employed in the art. Standard techniques are used for cloning, DNA and RNA isolation, amplification and purification. Generally enzymatic reactions involving DNA ligase, DNA polymerase, restriction endonucleases and the like are performed according to the manufacturer's specifications. These techniques and various other techniques are generally performed according to Sambrook et al., (1989).

The enzyme kinetics of the EPSPS enzymes used to produce glyphosate resistant cells need to demonstrate sufficient substrate binding activity (Kₘ PEP) and sufficient resistance to glyphosate inhibition (Kᵢ glyp) to function effectively in the present of glyphosate. The EPSPS enzyme can be assayed in vitro to demonstrate sufficient resistance to glyphosate inhibition. The assay is used to screen EPSPS enzymes for functionality in the presence of glyphosate. The absolute levels of Kₘ PEP and Kᵢ glyp, and the ratio between low Kₘ PEP and high Kᵢ glyp should be considered when determining the utility of the enzyme for engineering plants for glyphosate tolerance.

### Plant Recombinant DNA Constructs and Transformed Plants

A transgenic crop plant contains an exogenous polynucleotide molecule inserted into the genome of a crop plant cell. A crop plant cell, includes without limitation a plant cell further comprising suspension cultures, embryos, meristematic regions, callus tissue, leaves, roots, shoots, gametophytes, sporophytes, ovules, pollen and microspores, and seeds, and fruit. By “exogenous” it is meant that a polynucleotide molecule originates from outside the plant and that the polynucleotide molecule is inserted into the genome of the plant cell. An exogenous polynucleotide molecule can have a naturally occurring or non-naturally occurring polynucleotide sequence. One skilled in the art understands that an exogenous polynucleotide molecule can be a heterologous molecule derived from a different organism than the plant into which the polynucleotide molecule is introduced or can be a polynucleotide molecule derived from the same plant species as the plant into which it is introduced. The exogenous polynucleotide when expressed in a transgenic plant can provide an agronomically important trait.

The present invention provides a chimeric DNA molecule for producing-transgenic crop plants tolerant to glyphosate. Methods that are well known to those skilled in the art may be used to prepare the chimeric DNA molecule of the present invention. These methods include in vitro recombinant DNA techniques, synthetic techniques, and in vivo genetic recombination. For example, the techniques that are described in Sambrook et al., (1989). Exogenous polynucleotide molecules created by the methods may be transferred into a crop plant cell by Agrobacterium mediated transformation or other methods known to those skilled in the art of plant transformation.

Chimeric DNA molecules of the present invention are inserted into DNA constructs for propagation and transformation of plant cells. The DNA constructs are generally double Ti plasmid border DNA constructs that have the right border (RB or AGRtu.RB) and left border (LB or AGRtu.LB) regions of the Ti plasmid isolated from Agrobacterium tumefaciens comprising a T-DNA, that along with transfer molecules provided by the Agrobacterium cells, permits the integration of the T-DNA into the genome of a plant cell. The DNA constructs also contain the vector backbone DNA segments that provide replication function and antibiotic selection in bacterial cells, for example, an E. coli origin of replication such as ori322, a broad host range origin of replication such as oriV or oriRi, and a coding region for a selectable marker such as Spec/Strp that encodes for Tn7 aminoglycoside adenyltransferase (aadA) conferring resistance to spectinomycin or streptomycin, or a gentamicin (Gm, Gent) selectable marker gene. For plant transformation, the host bacterial strain is often Agrobacterium tumefaciens ABI, C58, or LBA4404, however, other strains known to those skilled in the art of plant transformation can function in the present invention.

In a preferred embodiment of the invention, a transgenic plant expressing a glyphosate resistant EPSPS is to be produced. Various methods for the introduction of the polynucleotide sequence encoding the EPSPS enzyme into plant cells are available and known to those of skill in the art and include, but are not limited to: (1) physical methods such as microinjection, electroporation, and microprojectile mediated delivery (Biolistics or gene gun technology); (2) virus mediated delivery methods; and (3) Agrobacterium-mediated transformation methods.

The most commonly used methods for transformation of a plant cell are: the Agrobacterium-mediated DNA transfer process and the Biolistics or microprojectile bombardment mediated process (such as, the gene gun). Typically, nuclear transformation is desired, but where it is desirable to specifically transform plastids, such as chloroplasts or amyloplasts, plant plastids may be transformed utilizing a microprojectile-mediated delivery of the desired polynucleotide.

Agrobacterium-mediated genetic transformation of plants involves several steps. The first step, in which the virulent Agrobaterium and plant cells are first brought into contact with each other, is generally called “inoculation”. Following the inoculation, the Agrobacterium and plant cells/tissues are permitted to be grown together for a period of several hours to several days or more under conditions suitable for growth and T-DNA transfer. This step is termed “co-culture”. Following co-culture and T-DNA delivery, the plant cells are treated with bactericidal or bacteriostatic agents to kill the Agrobacterium remaining in contact with the explant and/or in the vessel containing the explant. If this is done in the absence of any selective agents to promote preferential growth of transgenic versus non-transgenic plant cells, then this is typically referred to as the “delay” step. If done in the presence of selective pressure favoring transgenic plant cells, then it is referred to as a “selection” step. When a “delay” is used, it is typically followed by one or more “selection” steps.

With respect to microprojectile bombardment (U.S. Pat. No. 5,550,318; U.S. Pat. No. 5,538,880; U.S. Pat. No. 5,610,042), particles are coated with nucleic acids and delivered into cells by a propelling force. Exemplary particles include those comprised of tungsten, platinum, and preferably, gold. An illustrative embodiment of a method for delivering DNA into plant cells by acceleration is the Biolistics Particle Delivery System (BioRad, Hercules, Calif.), which can be used to propel particles coated with DNA or cells through a screen, such as a stainless steel or Nytex screen, onto a filter surface covered with monocot plant cells cultured in suspension.

The regeneration, development, and cultivation of plants from various transformed explants is well documented in the art. This regeneration and growth process typically includes the steps of selecting transformed cells and culturing those individualized cells through the usual stages of embryonic development through the rooted plantlet stage. Transgenic embryos and seeds are similarly regenerated. The resulting transgenic rooted shoots are thereafter planted in an appropriate plant growth medium such as soil. Cells that survive the exposure to the selective agent, or cells that have been scored positive in a screening assay, may be cultured in media that supports regeneration of plants. Developing plantlets are transferred to soil less plant growth mix, and hardened off, prior to transfer to a greenhouse or growth chamber for maturation.

The chimeric DNA molecules of the present invention can be used with any transformable cell or tissue. By transformable as used herein is meant a cell or tissue that is capable of further propagation to give rise to a plant. Those of skill in the art recognize that a number of plant cells or tissues are transformable in which after insertion of exogenous DNA and appropriate culture conditions the plant cells or tissues can form into a differentiated plant. Tissue suitable for these purposes can include but is not limited to immature embryos, scutellar tissue, suspension cell cultures, immature inflorescence, shoot meristem, nodal explants, callus tissue, hypocotyl tissue, cotyledons, roots, and leaves.

Plants that can be made to contain the chimeric DNA molecules of the present invention include, but are not limited to, Acacia, alfalfa, aneth, apple, apricot, artichoke, arugula, asparagus, avocado, banana, barley, beans, beet, blackberry, blueberry, broccoli, brussels sprouts, cabbage, canola, cantaloupe, carrot, cassaya, cauliflower, celery, cherry, cilantro, citrus, clementines, coffee, corn, cotton, cucumber, Douglas fir, eggplant, endive, escarole, eucalyptus, fennel, figs, forest trees, gourd, grape, grapefruit, honey dew, jicama, kiwifruit, lettuce, leeks, lemon, lime, Loblolly pine, mango, melon, mushroom, nut, oat, okra, onion, orange, an ornamental plant, papaya, parsley, pea, peach, peanut, pear, pepper, persimmon, pine, pineapple, plantain, plum, pomegranate, poplar, potato, pumpkin, quince, radiata pine, radicchio, radish, raspberry, rice, rye, sorghum, Southern pine, soybean, spinach, squash, strawberry, sugarbeet, sugarcane, sunflower, sweet potato, sweetgum, tangerine, tea, tobacco, tomato, turf, a vine, watermelon, wheat, yams, and zucchini.

The following examples are provided to better elucidate the practice of the present invention and should not be interpreted in any way to limit the scope of the present invention. Those skilled in the art will recognize that various modifications, additions, substitutions, truncations, etc., can be made to the methods and genes described herein while not departing from the spirit and scope of the present invention. Unless otherwise noted, terms are to be understood according to conventional usage by those of ordinary skill in the relevant art. Definitions of common terms in molecular biology may also be found in Rieger et al., Glossary of Genetics: Classical and Molecular, 5th edition, Springer-Verlag: New York, (1991); and Lewin, Genes V, Oxford University Press: New York, (1994). The nomenclature for DNA bases as set forth at 37 CFR § 1.822 is used. The standard one- and three-letter nomenclature for amino acid residues is used.

### EXAMPLES

### Example 1

### Isolation of EPSPS DNA Coding Sequences

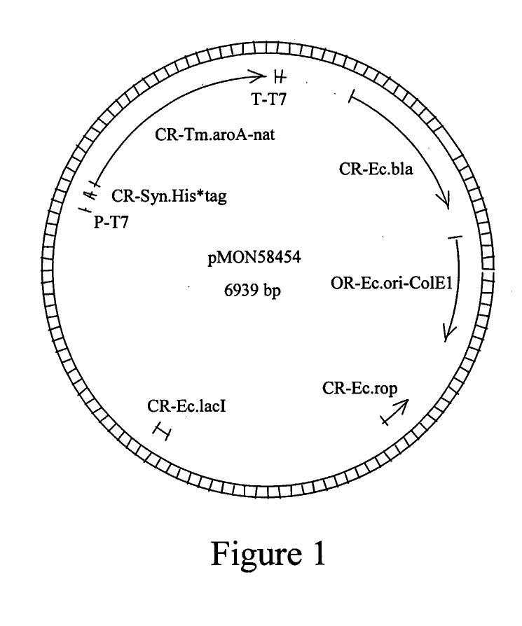

Thermatoga maritima (Tm) genomic DNA was obtained from the American Type Culture Collection (ATCC), Manassas, Va., accession #43589D. The genomic DNA was used as the template in PCR (High Fidelity PCR kit, Roche, Indianapolis, Ind.) to amplify the Tm EPSPS coding sequence using DNA primers. The DNA primers were designed based upon polynucleotide sequence of T. maritima EPSPS polynucleotide sequence (Genbank #Q9WYI0). PCR was set up in 2×50 μL (microliter) reactions as the following: dH₂O 80 μL; 10 mM dNTP 2 μL; 10× buffer 10 μL; genomic DNA (50 ng, nanogram) μL; Tm EPSPS 5′primer (SEQ ID NO: 42) (10 μM) 3 μL; Tm EPSPS 3′ primer (SEQ ID NO: 43) (10 μM) 3 μL; Enzyme 1 μL. PCR was carried out on a MJ Research PTC-200 thermal cycler (MJ Research, Waltham, Mass.) using the following program: Step 1 94° C. for 3 minutes; Step 2 94° C. for 20 seconds; Step 3 54° C. for 20 seconds; Step 4 68° C. for 20 seconds; Step 5 go to step 2, 30 times; Step 6 End. The PCR product was purified using QIAquick Gel Extraction kit (Qiagen Corp., Valencia, Calif.). The purified PCR product was digested with NdeI and PvuI and inserted by ligation into plasmid vector pET19b (Novagen, Madison, Wis.) by using Roche Rapid Ligation kit. The ligation product was transformed into competent E. coli DH5α using methods provided by the manufacturer (Stratagene Corp, La Jolla, Calif.). The pMON58454 (FIG. 1) plasmid DNA was purified from the transformed E. coli by the QIAprep Spin Miniprep kit (Qiagen Corp. Valencia, Calif.) and the insert confirmed by restriction enzyme analysis. The DNA sequence of the Tm EPSPS native (nat) coding sequence (CR-Tm.aroA-nat, SEQ ID NO: 28) from independent clones was produced and verified by standard DNA sequencing methods. The pMON58454 plasmid DNA containing the His-Tag verified Tm.aroA insert was transformed into BL21(DE3) pLysS strain (Stratagene, La Jolla, Calif.) for protein expression and purification using the methods provided by the manufacturer.

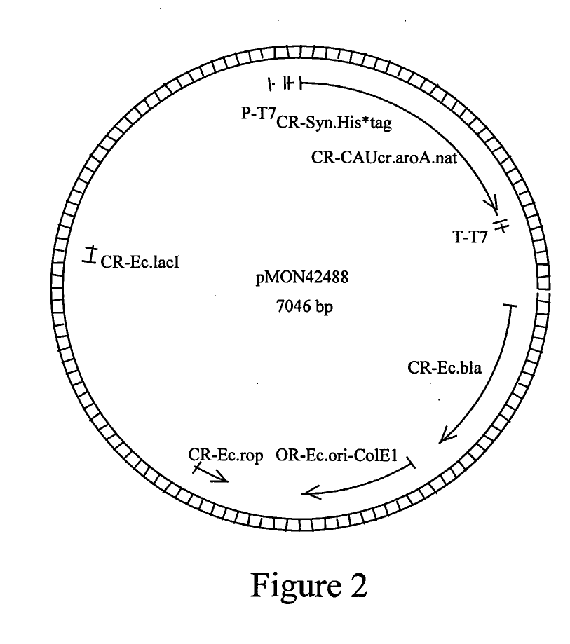

Genomic DNA of Caulobacter crescentus (Cc) (ATCC #19089D) was obtained from the ATCC. The genomic DNA was used as the template in a PCR to amplify the Cc EPSPS coding sequence. Oligonucleotide primers for PCR were designed based on sequences coding for the C. crescentus EPSPS (Genbank #AE006017). Restriction endonuclease recognition sites were incorporated at the 5′-end of the primers to facilitate cloning. The Long Temp PCR kit was purchased from Roche (Cat. No 1681834). PCR was set up in a 50 μL reaction as the following: dH₂O 40 μL; 2 mM dNTP 1 μL; 10× buffer 5 μL; DNA 1 μL (200-300 ng); Cc oligo-for (SEQ ID NO: 44) 1 μL; Cc oligo-rev (SEQ ID NO: 45) 1 μL; taq mix 1 μL. PCR was carried out on a MJ Research PTC-200 thermal cycler using the following program: Step 1 94° C. for 3 minutes; Step 2 94° C. for 20 seconds; Step 3 62° C. for 30 seconds; Step 4 68° C. for 90 seconds; Step 5 go to step 2, 30 times; Step 6 End. A fragment of the expected size of ˜1.3 kb was amplified from genomic DNA. The PCR fragment was purified using Qiagen Gel Purification kit (Cat. No 28104). The purified PCR fragment was digested with the restriction enzymes NdeI and XhoI, and inserted by ligation into plasmid pET19b (Novagen) that was digested with the same enzymes. The ligation mixture was used to transform the competent E. coli strain DH5α (Invitrogen, Carlsbad, Calif.) following the manufacturer's instructions. The transformed cells were plated on a Petri dish containing carbenicillin at a final concentration of 0.1 mg/mL. The plate was then incubated at 37° C. overnight. Single colonies were picked the next day and used to inoculate a 3 mL liquid culture containing 0.1 mg/mL ampicillin. The liquid culture was incubated overnight at 37° C. with agitation at 250 rpm. Plasmid DNA was prepared from 1 mL of the liquid culture using Qiagen miniprep Kit (Cat. No. 27160). The DNA was eluted in 50 μL of deionized H₂O. The DNA sequence of the Cc EPSPS native (nat) coding sequence (CR-CAUcr.aroA-nat, SEQ ID NO: 23) from independent clones was produced and verified by standard DNA sequencing methods. The pMON42488 (FIG. 2) plasmid DNA from the verified clone was transformed into BL21(DE3) pLysS strain for protein expression and purification following the manufacturers instructions.

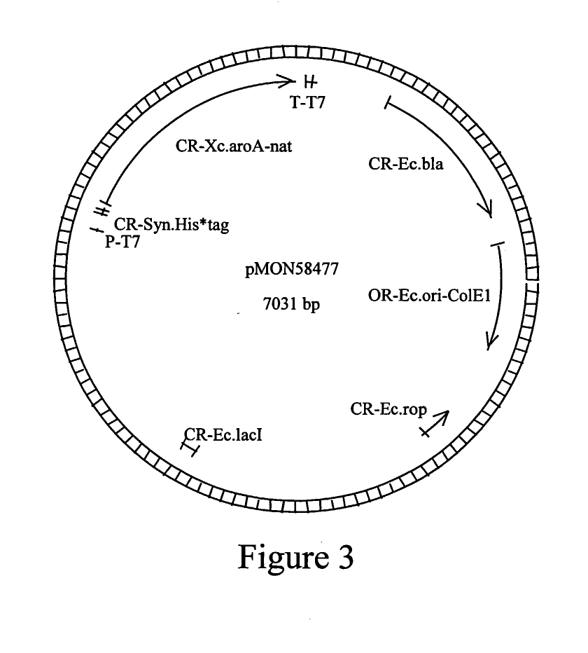

Genomic DNA of Xanthomonas campestris (Xc) (ATCC #33913D) was obtained from the ATCC. The genomic DNA was used as the template in a PCR to amplify the XC EPSPS coding sequence Oligonucleotide primers for PCR were designed based on X. campestris EPSPS coding sequence (Genbank #XAN202351). Restriction endonuclease recognition sites were incorporated at the 5′-end of the primers to facilitate cloning. The SuperMix High Fidelity PCR kit was purchased from Invitrogen (Cat. No 10790-020). PCR was set up in a 50 μL reaction as the following: SuperMix buffer 45 μL; DNA 1 μL (75-200 ng); 10 μM Xancp-A1F (SEQ ID NO: 46) 1 μL; 10 μM Xancp-A1R (SEQ ID NO: 47) 1 μL. PCR was carried out on a MJ Research PTC-200 thermal cycler using the following program: Step 1 94° C. for 2 minutes; Step 2 94° C. for 20 seconds; Step 3 56° C. for 30 seconds; Step 4 68° C. for 1 minute 40 seconds; Step 5 go to step 2, 30 times; Step 6 End. A fragment of the expected size of ˜1.3 kb was amplified from genomic DNA. The PCR fragment in 4 μl PCR reaction was inserted into Invitrogen's Zero Blunt TOPO vector (Cat. #K2800-20) and transformed into E. coli strain DH5α (Invitrogen). Single colonies were picked the next day and used to inoculate a 3 mL liquid culture containing 0.5 mg/mL kanamycin. The liquid culture was incubated overnight at 37° C. with agitation at 250 rpm. Plasmid DNA was prepared from 1 mL of the liquid culture using Qiagen miniprep Kit (Cat. No. 27160). The DNA was eluted in 50 μL of H₂O. The entire coding region (CR-) of nineteen independent clones were sequenced by and verified by standard DNA sequencing methods. The PCR fragment on TOPO vector with confirmed sequence (CR-Xc.aroA-nat, SEQ ID NO: 20) was then digested with the restriction enzymes NdeI and XhoI, and inserted by ligation into plasmid pET19b (Novagen) that was digested with the same enzymes. The pMON58477 (FIG. 3) plasmid DNA from the verified clone was transformed into BL21(DE3)pLysS strain for protein expression and purification following the manufacturers instructions.

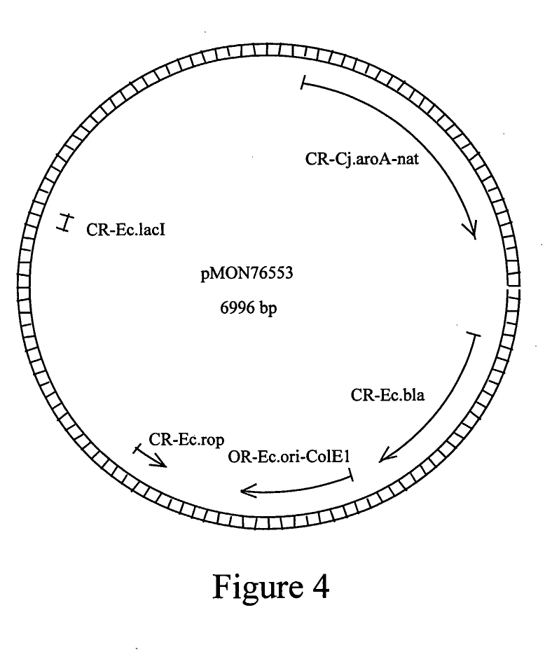

Genomic DNA from Campylobacter jejuni (Cj) was obtained from the ATCC (#700819D). The EPSPS coding sequence was isolated using a PCR based DNA amplification method and DNA primers. The High Fidelity PCR kit from Roche was used. The primers were designed based on published sequence of the C. jejuni EPSPS coding sequence (Genbank #CJU10895). PCR was set up in 2×50 μL reactions as the following: dH₂O 80 μL; 10 mM dNTP 2 μL; 10× buffer 10 μL; genomic C. jejuni DNA (50 ng) μL; CampyEPSPS 5′primer (SEQ ID NO: 48) (10 μM) 3 μL; CampyEPSPS 3′ primer (SEQ ID NO: 49) (10 μM) 3 μL; Enzyme 1 μL. PCR was carried out on a MJ Research PTC-200 thermal cycler (MJ Research) using the following program: Step 1 94° C. for 3 minutes; Step 2 94° C. for 20 seconds; Step 3 54° C. for 20 seconds; Step 4 68° C. for 20 seconds; Step 5 go to step 2, 30 times; Step 6 End. The PCR product was purified using QIAquick Gel Extraction kit (Qiagen Corp.). The purified PCR product was digested with NdeI and PvuI and inserted by ligation into plasmid vector pET19b (Novagen,) by using Roche Rapid Ligation kit. The ligation product was transformed into competent E. coli DH5α (Stratagene). The pMON76553 (FIG. 4) plasmid DNA was purified from the transformed E. coli by the QIAprep Spin Miniprep kit (Qiagen Corp.) and the insert confirmed by restriction enzyme analysis. The DNA sequence of the Cj EPSPS native coding sequence (CR-Cj.aroA-nat, SEQ ID NO: 32) from independent clones was produced and verified by standard DNA sequencing methods. The pMON76553 (FIG. 4) plasmid DNA from the verified clone was transformed into BL21(DE3)pLysS strain for protein expression and purification.

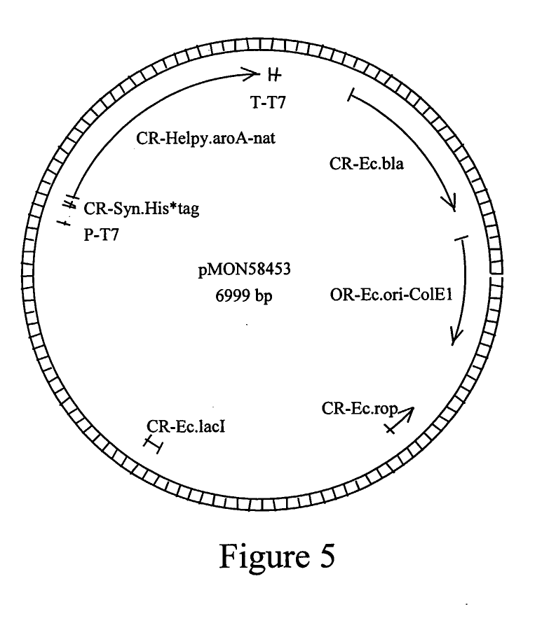

Genomic DNA from Helicobacter pylori (Hp) was obtained from the ATCC (accession #700392D). The EPSPS coding sequence was isolated using a PCR based DNA amplification method and DNA primers designed from the DNA sequence of EPSPS found in Genbank #HP0401. The High Fidelity-PCR kit from Roche was used and the PCR conditions described for the isolation of the H. pylori. EPSPS coding sequence. The DNA primers used were HelpyEPSPS 5′ (SEQ ID NO: 50) and HelpyEPSPS 3′(SEQ ID NO: 51). The purified PCR product was digested with NdeI and PvuI and inserted by ligation into plasmid vector pET19b (Novagen) by using Roche Rapid Ligation kit. The ligation product was transformed into competent E. coli DH5α (Stratagene). The pMON58453 (FIG. 5) plasmid DNA was purified from the transformed E. coli by the QIAprep Spin Miniprep kit (Qiagen Corp.) and the insert confirmed by restriction enzyme analysis. The DNA sequence of the HpEPSPS native coding sequence (CR-Helpy.aroA-nat, SEQ ID NO: 31) from independent clones was produced and verified by standard DNA sequencing methods. The pMON58453 plasmid DNA from the verified clone was transformed into BL21(DE3)pLysS strain for protein expression and purification.

### Example 2

### EPSPS Enzyme Expression and Activity Assays

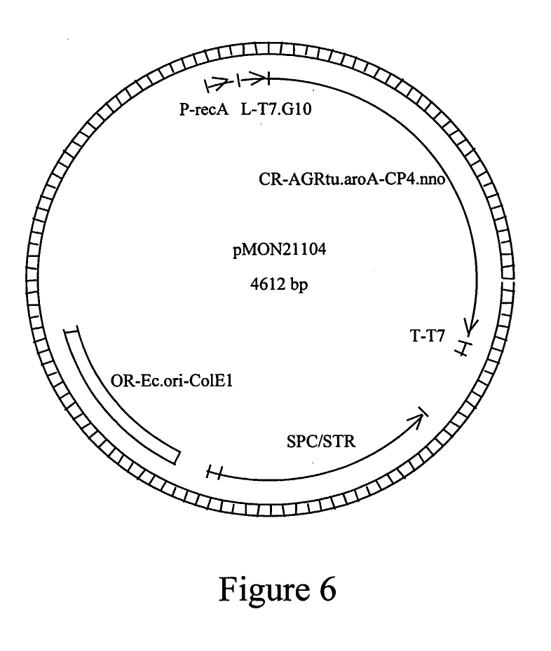

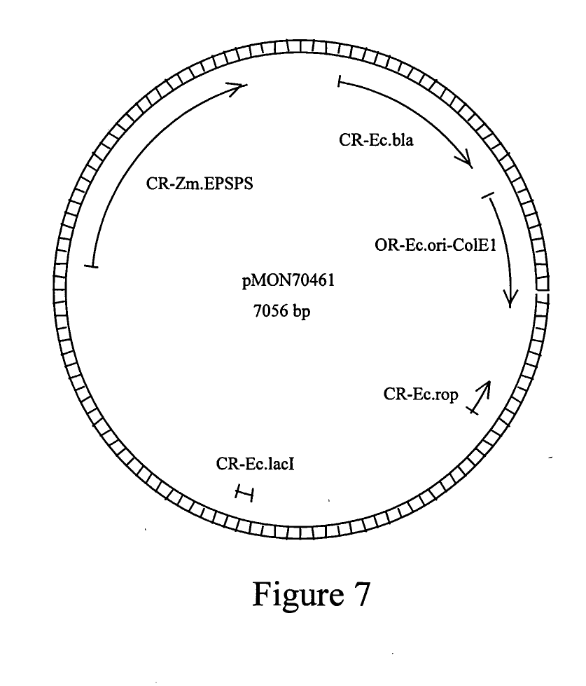

Plasmid DNA containing the EPSPS coding sequence (FIG. 1. pMON58454, T. maritima EPSPS(CR-Tm.aroA-nat); FIG. 2. pMON42488, C. crescentus EPSPS(CR-CAUcr.aroA.nat); FIG. 3. pMON58477, X. campestris EPSPS(CR-Xc.aroA-nat); FIG. 4. pMON76553, C. jejuni EPSPS(CR-Cj.aroA-nat); FIG. 5. pMON58453H. pylori EPSPS(CR-Helpy.aroA-nat); FIG. 6. pMON21104 A. tumefaciens CP4 EPSPS(CR-AGRtu.aroA-CP4.nno), and FIG. 7. pMON70461 Zea mays EPSPS(CR-Zm.EPSPS) are contained in BL21trxB (DE3) pLysS strain for protein expression and purification.

The EPSPS proteins were expressed from the chimeric DNA molecules that contained the coding sequences for the EPSPS enzymes, and were partially purified using the protocols outlined in the pET system manual ninth edition (Novagen). A single colony or a few microliters (μL) from a glycerol stock was inoculated into 4 mL (milliliter) Luria Broth (LB) medium containing 0.1 mg/mL (milligram/milliliter) carbenicillin. The culture was incubated with shaking at 37° C. for 4 hours. The cultures were stored at 4° C. overnight. The following morning, 1 mL of the overnight culture was used to inoculate 100 mL of fresh LB medium containing 0.1 mg/mL carbenicillin. The cultures were incubated with shaking at 37° C. for 4-5 hours then the cultures were placed at 4° C. for 5-10 minutes. The cultures were then induced with IPTG (NAME, 1 mM (millimolar) final concentration) and incubated with shaking at −30° C. for 4 hours or 20° C. overnight. The cells were harvested by centrifugation at 7000 rpm (revolutions per minute) for 20 minutes at 4° C. The supernatant was removed and the cells were frozen at −70° C. until further use. The proteins were extracted by resuspending the cell pellet in BugBuster reagent (Novagen) using 5 mL reagent per gram of cells. Benzonase (125 Units, Novagen) was added to the resuspension and the cell suspension was then incubated on a rotating mixer for 20 minutes at room temperature. The cell debris was removed by centrifugation at 10,000 rpm for 20 minutes at room temperature. The supernatant was passed through a 0.45 μm (micrometer) syringe-end filter and transferred to a fresh tube. A pre-packed column containing 1.25 mL of His-Bind resin was equilibrated with 10 mL of 5 mM imidazole, 0.5 M NaCl, 20 mM Tris-HCl pH 7.9 (1× Binding buffer). The column was then loaded with the prepared cell extract. After the cell extract had drained, the column was then washed with 10 mL of 1× Binding buffer, followed with 10 mL of 60 mM imidazole, 0.5 M NaCl, 20 mM Tris-HCl pH 7.9 (1× Wash buffer). The protein was eluted with 5 mL of 1 M imidazole, 0.5 M NaCl, 20 mM Tris-HCl pH 7.9 (1× elution buffer). Finally, the protein was dialyzed into 50 mM Tris-HCl pH 6.8. The resulting protein solution was concentrated to ˜0.1-0.4 mL using Ultrafree centrifugal device (Biomax-10K MW cutoff, Millipore Corp., Beverly, Mass.). Proteins were diluted to 10 mg/mL and 1 mg/mL in 50 mM Tris pH 6.8, 30% final glycerol and stored at −20° C. Protein concentration was determined using Bio-Rad protein assay (Bio-Rad Laboratories, Hercules, Calif.). BSA was used to generate a standard curve 1-5 μg (microgram). Samples (10 μL) were added to wells in a 96 well-plate and mixed with 200 μL of Bio-Rad protein assay reagent (1 part dye reagent concentrate:4 parts water). The samples were read at OD₅₉₅ after ˜5 minutes using a SpectraMAX 250 plate reader (Molecular Devices Corporation, Sunnyvale, Calif.) and compared to the standard curve.

EPSPS enzyme assays contained 50 mM K⁺-HEPES pH 7.0 and 1 mM shikimate-3-phosphate (Assay mix). The Kₘ-PEP were determined by incubating assay mix (30 μL) with enzyme (10 μL) and varying concentrations of [¹⁴C]PEP in a total volume of 50 μL. The reactions were quenched after various times with 50 μL of 90% ethanol/0.1 M acetic acid pH 4.5 (quench solution). The samples were centrifuged at 14,000 rpm and the resulting supernatants were analyzed for ¹⁴C-EPSP production by HPLC. The percent conversion of ¹⁴C-PEP to ¹⁴C-EPSP was determined by HPLC radioassay using an AX100 weak anion exchange HPLC column (4.6×250 mm, SynChropak) with 0.26 M isocratic potassium phosphate eluant, pH 6.5 at 1 mL/min mixed with Ultima-Flo AP cocktail at 3 mL/min (Packard). Initial velocities were calculated by multiplying fractional turnover per unit time by the initial concentration of the substrate.

The inhibition constant (Kᵢ) was determined by incubating assay mix (30 μL) with and without glyphosate and ¹⁴C-PEP (10 μL of 2.6 mM). The reaction was initiated by the addition of enzyme (10 μL). The assay was quenched after 2 minutes with quench solution. The samples were centrifuged at 14,000 rpm and the conversion of ¹⁴C-PEP to ¹⁴C-EPSP was determined as shown above. The steady-state and IC₅₀ data were analyzed using the GraFit software (Erithacus Software, UK). The Kᵢ value was calculated from the IC₅₀ values using the equation Kᵢ=[IC]₅₀/(1+[S]/Kₘ). The assays were done such that the ¹⁴C-PEP to ¹⁴C-EPSP turnover was <30%. In these assays bovine serum albumin (BSA) and phosphoenolpyruvate were obtained from Sigma. Phosphoenol-[1-¹⁴C]pyruvate (29 mCi/mmol) was from Amersham Corp., Piscataway, N.J.

The results of the EPSPS enzyme analysis are shown in Table 2. The kinetic parameters of the EPSPS enzymes of the present invention are compared to the class II CP4 EPSPS and class I wild type maize EPSPS (WT maize). All of the EPSPS enzymes have a Kₘ-PEP equal to or better than the endogenous WT maize enzyme and all are resistant to glyphosate relative to this class I enzyme. Additionally, the low Kₘ-PEP of some of the EPSPS enzymes may be useful to enhance the flux of substrate in the shikimate acid biosynthesis pathway thereby providing an increase in the products of the pathway.

TABLE 2
EPSPS Steady-state kinetic parameters

| Enzyme* | Kₘ-PEP (μM) | Kᵢ (μM) | Kᵢ/Kₘ |
|---|---|---|---|
| CP4 EPSPS | 14.4 | 5100 | 354.2 |
| C. crescentus | 2.0 | 140.6 | 70.3 |
| (SEQ ID NO: 9) | | | |
| T. maritima | 1.4 | 900 | 643 |
| (SEQ ID NO: 14) | | | |
| H. pylori | 2.1 | 12.9 | 6.1 |
| (SEQ ID NO: 17) | | | |
| C. jejuni | 7.4 | 22.4 | 3.0 |
| (SEQ ID NO: 18) | | | |
| X. campestris | 27.6 | 2500 | 90.6 |
| (SEQ ID NO: 6) | | | |
| WT maize | 27 | 0.5 | 0.02 |

### Example 3

### Plant Chimeric DNA Constructs

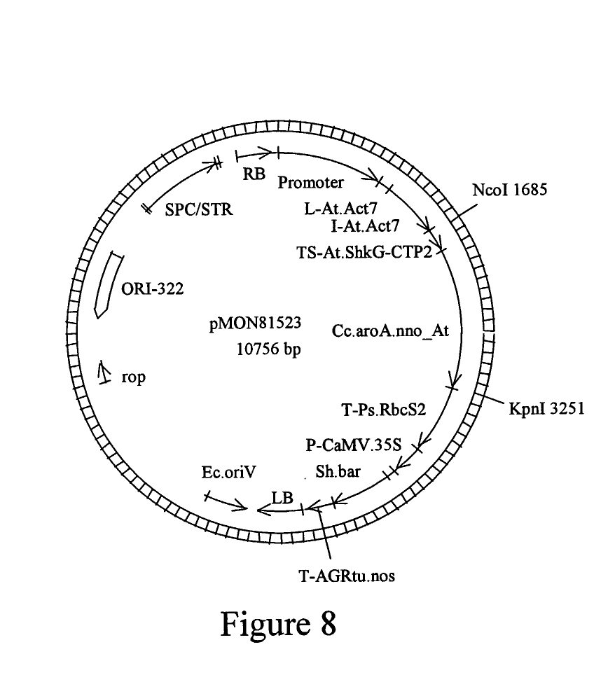

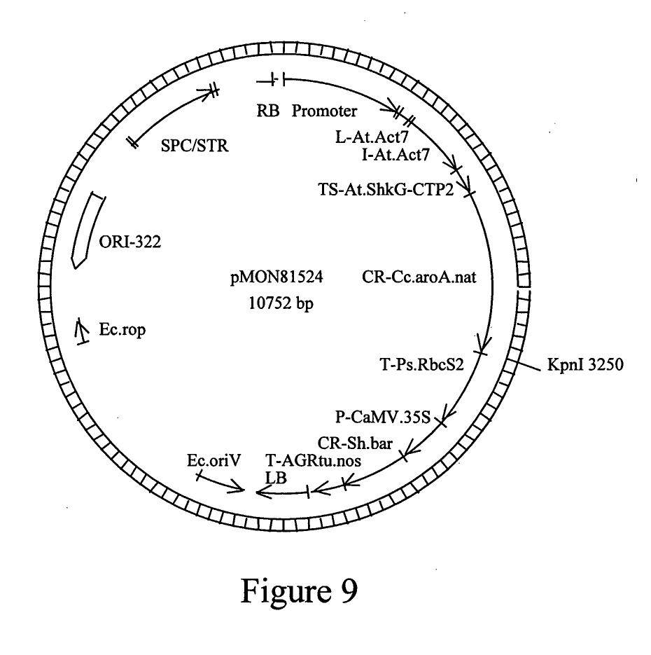

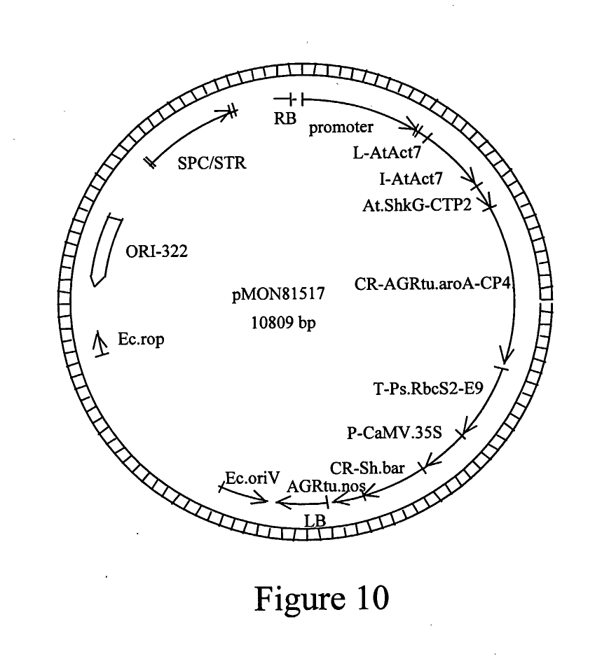

The DNA molecules encoding the EPSPS proteins of the present invention are made into plant expression DNA constructs for transformation into plant cells. For example, the chimeric DNA constructs: pMON81523 (FIG. 8) and pMON81524 (FIG. 9) contain a plant expression cassette comprising the regulatory elements of a promoter molecule, a leader molecule (L-At.Act7, Arabidopsis thaliana Act7 leader DNA molecule) and an intron molecule (I-At.Act7, Arabidopsis thaliana Act7 intron DNA molecule) that function in plants to provide sufficient expression of an operably linked chimeric CTP-EPSPS coding sequence linked to a 3′ transcriptional termination region. The chimeric TS-At.ShkG-CTP2-Cc.aroA.nno-At DNA molecule is contained on an NcoI/KpnI DNA fragment in pMON81523. The TS-At.ShkG-CTP2 DNA molecule encodes for the Transit Signal (TS) isolated from the Arabidopsis thaliana ShkG gene, also referred to as At.CTP2 (Klee et al., Mol. Gen. Genet. 210:47-442, 1987). The Cc.aroA.nno-At is an artificial polynucleotide encoding the C. crescentus EPSPS protein, the artificial polynucleotide (SEQ ID NO: 34) is designed for enhanced expression in plant cells using an Arabidopsis thaliana (At) or Glycine max (Gm) usage table (for example, those tables illustrated in WO04009761) that is a modification of the native polynucleotide sequence isolated from C. crescentus (SEQ ID NO: 23). The Termination region (T-) is the pea (Pisum sativum, Ps) ribulose 1,5-bisphosphate carboxylase (referred to as E9 3′ or T-Ps.RbcS, Coruzzi, et al., EMBO J. 3:1671-1679, 1984). Also contained in pMON81523 is a plant expression cassette that provides a selectable marker gene for selection of transgenic plant cells using glufosinate herbicide, this is the P-CaMV.35S/Sh.bar coding region/T-AGRtu.nos. The plant expression cassettes are flanked by an Agrobacterium tumefaciens Ti plasmid right border (RB) and left border (LB) DNA regions. The plant chimeric DNA construct pMON81524 contains the same regulatory elements operably linked DNA molecules as pMON81523 except that the Cc.aroA.nat polynucleotide (SEQ ID NO: 23) is used, this is the native C. crescentus polynucleotide molecule. For comparative purposes, the plant chimeric DNA construct pMON81517 (FIG. 10) contains the same operably linked DNA molecules as pMON81523 and pMON81524, except that the Agrobacterium tumefaciens strain CP4 EPSPS coding sequence (AGRtu.aroA-CP4) is used in place of the C. crescentus polynucleotides. The transfer DNA of these DNA constructs is inserted into the genome of plant cells, for example, Arabidopsis and tobacco cells by an Agrobacterium-mediated transformation method to provide transgenic glyphosate tolerant plants.

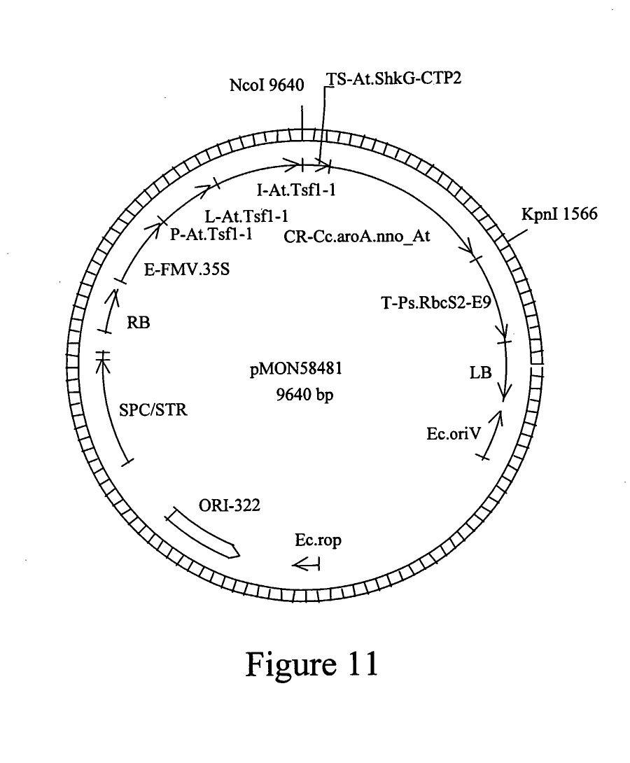

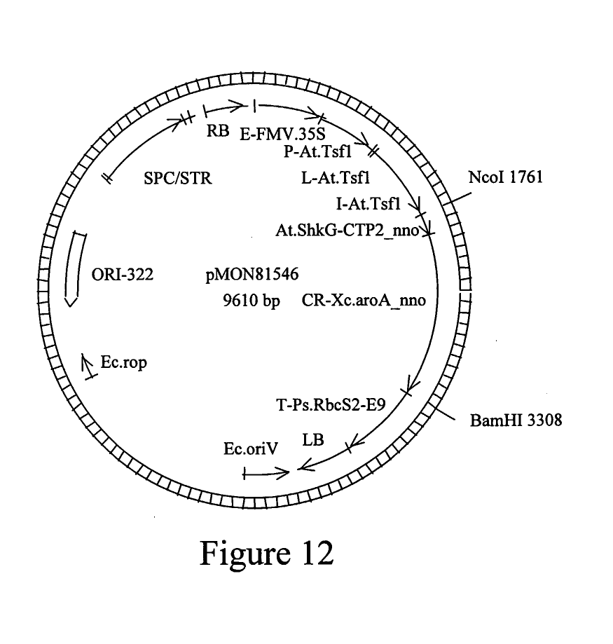

Additional plant chimeric DNA constructs are made that contain the Cc.aroA.nno-At polynucleotide (pMON58481, FIG. 11) and the X. campestris artificial polynucleotide (SEQ ID NO: 35) Xc.aroA.nno-At (pMON81546, FIG. 12). The regulatory genetics elements driving expression of these polynucleotides are the chimeric promoter (P-FMV.35S-At.Tsf1), leader (L-At.Tsf1) and intron (I-At.Tsf1) (U.S. Pat. No. 6,660,911, SEQ ID NO:28) and the T-Ps.RbcS2 termination region. The Xc.aroA.nno-At is an artificial polynucleotide encoding the X. campestris EPSPS protein, the artificial polynucleotide (SEQ ID NO: 35) is designed for enhanced expression in plant cells using an Arabidopsis thaliana codon usage table (for example, WO04009761, Table 2) that modifies the native polynucleotide sequence isolated from X. camnpestris (SEQ ID NO: 20). The transfer DNA of these DNA constructs is inserted into the genome of a plant cell by an Agrobacterium-mediated transformation method, for example, a soybean cell to provide transgenic glyphosate tolerant soybean plants.

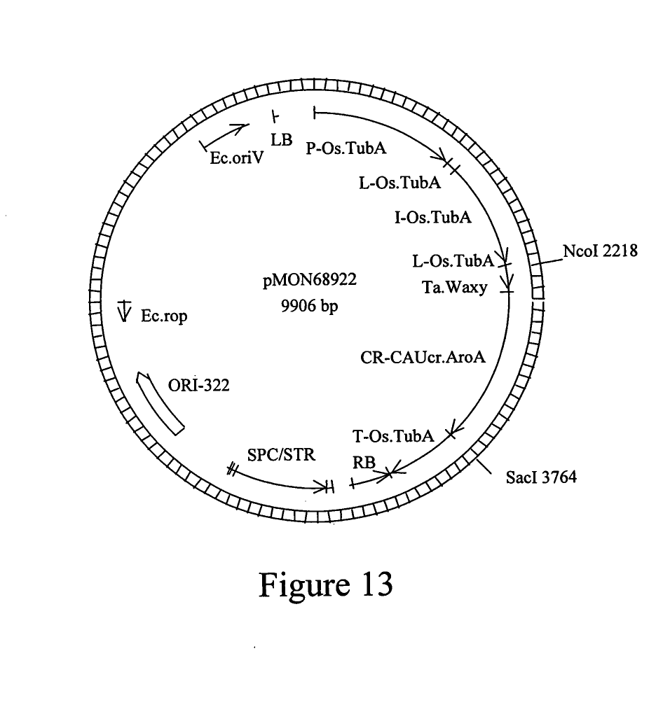

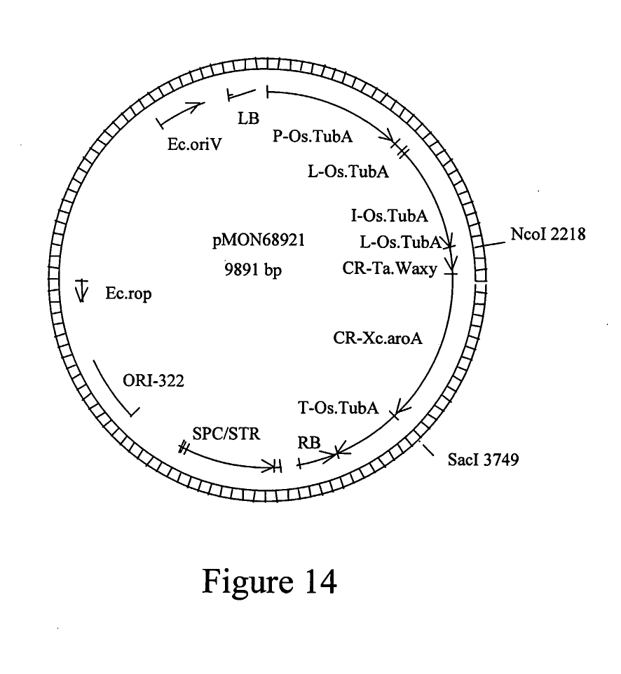

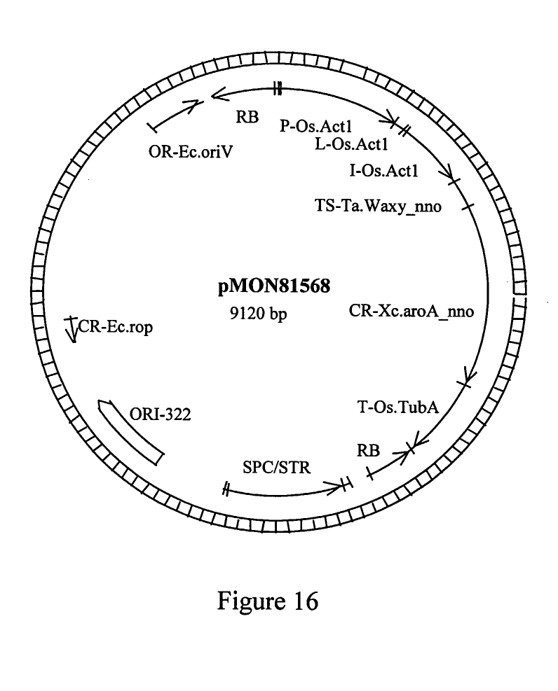

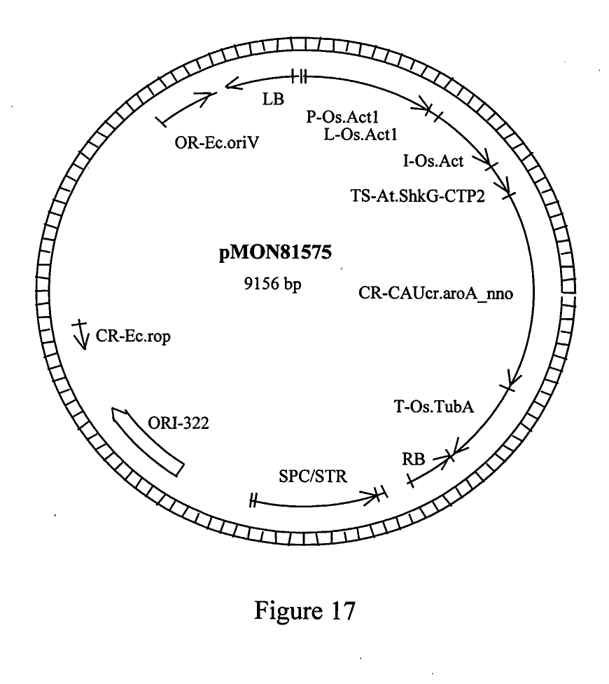

Chimeric plant DNA constructs can be designed for expression in monocot plant cells. For example, pMON68922 (FIG. 13) and pMON68921 (FIG. 14) contain plant expression cassettes and regulatory elements and coding sequences for expression in monocot cells. Additionally, the DNA of the C. crescentus EPSPS and X. campestris EPSPS coding sequences are modified for enhanced expression in monocot cells. The Xc.aroA.nno-mono is an artificial polynucleotide encoding the X. campestris EPSPS protein, the artificial polynucleotide (SEQ ID NO: 37) is designed for enhanced expression in plant cells using a monocot codon usage table (for example, WO04009761, Table 3) that modifies the native polynucleotide sequence isolated from X. campestris (SEQ ID NO: 20). The Cc.aroA.nno-mono is an artificial polynucleotide encoding the C. crescentus EPSPS protein, the artificial polynucleotide (SEQ ID NO: 36) is designed for enhanced expression in plant cells using a monocot codon usage table (for example, WO04009761, Table 3) that modifies the native polynucleotide sequence isolated from C. crescentus (SEQ ID NO: 23). The regulatory elements of pMON68921 (FIG. 14), pMON68922 (FIG. 13), pMON81568 (FIG. 16) and pMON81575 (FIG. 17) comprise promoter (P-), leader (L-), intron (I-), (TS-) transit signal, and termination (T-) DNA molecules. In these examples, the regulatory elements are isolated rice tubulin A gene elements, and are illustrated in these DNA constructs as P-Os.TubA, L-Os.TubA, I-Os.TubA and T-Os.TubA or from rice actin 1 gene elements and are illustrated in these DNA constructs as P-Os.Act1, L-Os.Act1, and I-Os.Act1. A DNA molecule encoding a CTP isolated from the wheat-GBSS coding sequence (Genbank X57233), herein referred to as TS-Ta.Wxy, is modified then fused to the Xc.aroA.nno-mono polynucleotide to create a chimeric DNA molecule (SEQ ID NO: 40) and also fused to the Cc.aroA.nno-mono to create a chimeric DNA molecule (SEQ ID NO: 41), these DNA molecules are operably linked in pMON68921 and pMON68922, respectively. The transfer DNA of these DNA constructs is inserted into the genome of a plant cell by an Agrobacterium-mediated transformation method, for example, a corn cell to provide transgenic glyphosate tolerant corn plants.

### Example 4

### Plant Transformation

Arabidopsis embryos have been transformed by an Agrobacterium mediated method described by Bechtold N, et. al., CR Acad Sci Paris Sciences di la vie/life sciences 316: 1194-1199, (1993). This method has been modified for use with the constructs of the present invention to provide a rapid and efficient method to transform Arabidopsis and select for a glyphosate tolerant phenotype

An Agrobacterium strain ABI containing a chimeric DNA construct, such as pMON81523, pMON81524, and pMON81517, is prepared as inoculum by growing in a culture tube containing 10 mls Luria Broth and antibiotics, for example, 1 ml/L each of spectinomycin (100 mg/ml), chloramphenicol (25 mg/ml), kanamycin (50 mg/ml) or the appropriate antibiotics as determined by those skilled in the art. The culture is shaken in the dark at 28° C. for approximately 16-20 hours.

The Agrobacterium inoculum is pelleted by centrifugation and resuspended in 25 ml Infiltration Medium (MS Basal Salts 0.5%, Gamborg's B-5 Vitamins 1%, Sucrose 5%, MES 0.5 g/L, pH 5.7) with 0.44 nM benzylaminopurine (10 ul of a 1.0 mg/L stock in DMSO per liter) and 0.02% Silwet L-77 to an OD₆₀₀ of 0.6.

Mature flowering Arabidopsis plants are vacuum infiltrated in a vacuum chamber with the Agrobacterium inoculum by inverting the pots containing the plants into the inoculum. The chamber is sealed, a vacuum is applied for several minutes, release the vacuum suddenly, blot the pots to remove excess inoculum, cover pots with plastic domes and place pots in a growth chamber at 21° C. 16 hours light and 70% humidity. Approximately 2 weeks after vacuum infiltration of the inoculum, cover each plant with a Lawson 511 pollination bag. Approximately 4 weeks post infiltration, withhold water from the plants to permit dry down. Harvest seed approximately 2 weeks after dry down.

The transgenic Arabidopsis plants produced by the infiltrated seed embryos are selected from the nontransgenic plants by a germination selection method. The harvested seed is surface sterilized then spread onto the surface of selection media plates containing MS Basal Salts 4.3 g/L, Gamborg B-5 (500×) 2.0 g/L, Sucrose 10 g/L, MES 0.5 g/L, and 8 g/L Phytagar with Carbenicillin 250 mg/L, Cefotaxime 100 mg/L, and PPM 2 ml/L and appropriate selection agent added as a filter sterilized liquid solution, after autoclaving. The selection agent can be an antibiotic or herbicide, for example kanamycin 60 mg/L, glyphosate 40-60 μM, or bialaphos 10 mg/L are appropriate concentrations to incorporate into the media depending on the DNA construct and the plant expression cassettes contained therein that are used to transform the embryos. When using glyphosate selection, the sucrose is deleted from the basal medium. Put plates into a box in a 4° C. to allow the seeds to vernalize for ˜2-4 days. After seeds are vernalized, transfer to a growth chamber with cool white light bulbs at a 16/8 light/dark cycle and a temperature of 23 C. After 5-10 days at −23° C. and a 16/8 light cycle, the transformed plants will be visible as green plants. After another 1-2 weeks, plants will have at least one set of true leaves. Transfer plants to soil, cover with a germination dome, and move to a growth chamber, keep covered until new growth is apparent, usually 5-7 days.

### Tobacco Transformation

An Agrobacterium strain ABI containing a chimeric DNA construct, such as pMON81523, pMON81524, and pMON81517, is prepared as inoculum by growing in a culture tube containing 10 mls Luria Broth and antibiotics, for example, 1 ml/L each of spectinomycin (100 mg/ml), chloramphenicol (25 mg/ml), kanamycin (50 mg/ml) or the appropriate antibiotics as determined by those skilled in the art. The culture is shaken in the dark at 28° C. for approximately 16-20 hours.

Tobacco transformation is performed as follows: stock tobacco plants maintained by in-vitro propagation. Stems are cut into sections and placed into phytatrays. Leaf tissue is cut and placed onto solid pre-culture plates of MS104 to which 2 mls of liquid TXD medium (Table 3. Basal Media Recipes) and a sterile Whatman filter disc have been added. Pre-culture the explants in warm room (230 Celsius, continuous light) for 1-2 days. The day before inoculation, a 10 μl loop of a transformed Agrobacterium containing one of the DNA constructs is placed into a tube containing 10 mls of YEP media with appropriate antibiotics to maintain selection of the DNA construct. The tube is put into a shaker to grow overnight at 28° C. The OD₆₀₀ of the Agrobacterium is adjusted to 0.15-0.30 OD₆₀₀ with TXD medium. Inoculate tobacco leaf tissue explants by pipetting 7-8 mls of the liquid Agrobacterium suspension directly onto the pre-culture plates covering the explant tissue. Allow the Agrobacterium to remain on the plate for 15 minutes. Tilt the plates and aspirate liquid off using a sterile 10 ml wide bore pipette. The explants are co-cultured on these same plates for 2-3 days. The explants are then transferred to MS104 containing these additives, added post autoclaving: 500 mg/L carbenicillin, 100 mg/L cefotoxime, 150 mg/L vanamycin and 300 mg/L kanamycin. At 3-4 weeks, callus is transferred to fresh kanamycin containing medium. At 6-8 weeks shoots should be excised from the callus and cultured on MS0+500 mg/L carbenicillin+100 mg/L kanamycin media and allowed to root. Rooted shoots are then transferred to soil after 2-3 weeks.

TABLE 3
Basal Medium Recipes

MS0
4.4 g MS B-5
30 g sucrose
9 g Sigma TC agar
MS104
4.4 g MS basal salts + B5 vitamins
30 g sucrose
1.0 mg BA
0.1 mg NAA
9 g Sigma TC agar
TXD
4.3 g Gibco MS
2 ml Gamborg's B-5 500X
8 ml pCPA(.5 mg/ml)
.01 ml kinetin(.5 mg/ml)
30 g sucrose

### Soybean Transformation

The DNA constructs, pMON58481 and pMON81546 were transformed into soybean cells essentially as described in U.S. Pat. No. 5,569,834 and U.S. Pat. No. 5,416,011 herein incorporated by reference in its entirety.

### Corn Transformation

The chimeric DNA constructs comprising the EPSPS coding sequences of the present invention are transformed into corn plant cells by an Agrobacterium-mediated transformation method. For example, a disarmed Agrobacterium strain C58 harboring a binary DNA construct of the present invention is used. The DNA construct is transferred into Agrobacterium by a triparental mating method (Ditta et al., Proc. Natl. Acad. Scd. 77:7347-7351, 1980). Liquid cultures of Agrobacterium containing pMON68922 or pMON68921 are initiated from glycerol stocks or from a freshly streaked plate and grown overnight at 26° C.-28° C. with shaking (approximately 150 revolutions per minute, rpm) to mid-log growth phase in liquid LB medium, pH 7.0, containing 50 mg/l (milligram per liter) kanamycin, and either 50 mg/l streptomycin or 50 mg/l spectinomycin, and 25 mg/l chloramphenicol with 200 μM acetosyringone (AS). The Agrobacterium cells are resuspended in the inoculation medium (liquid CM4C, as described in Table 8 of U.S. Pat. No. 6,573,361) and the cell density is adjusted such that the resuspended cells have an optical density of 1 when measured in a spectrophotometer at a wavelength of 660 nm (i.e., OD₆₆₀). Freshly isolated Type II immature HIIxLH198 and HiII corn embryos are inoculated with Agrobacterium and co-cultured 2-3 days in the dark at 23° C. The embryos are then transferred to delay media (N6 1-100-12; as described in Table 1 of U.S. Pat. No. 5,424,412) supplemented with 500 mg/l Carbenicillin (Sigma-Aldrich, St Louis, Mo.) and 20 μM AgNO₃) and incubated at 28° C. for 4 to 5 days. All subsequent cultures are kept at this temperature.

The corn coleoptiles are removed one week after inoculation. The embryos are transferred to the first selection medium (N61-0-12, as described in Table 1 of U.S. Pat. No. 5,424,412), supplemented with 500 mg/l carbenicillin and 0.5 mM glyphosate. Two weeks later, surviving tissues are transferred to the second selection medium (N61-0-12) supplemented with 500 mg/l carbenicillin and 1.0 mM glyphosate. Surviving callus is subcultured every 2 weeks for about 3 subcultures on 1.0 mM glyphosate. When glyphosate tolerant tissues are identified, the tissue is bulked up for regeneration. For regeneration, callus tissues are transferred to the regeneration medium (MSOD, as described in Table 1 of U.S. Pat. No. 5,424,412) supplemented with 0.1 μM abscisic acid (ABA; Sigma-Aldrich, St Louis, Mo.) and incubated for two-weeks. The regenerating calli are transferred to a high sucrose medium and incubated for two weeks. The plantlets are transferred to MSOD media (without ABA) in a culture vessel and incubated for two weeks. Then the plants with roots are transferred into soil. Plants can be treated with glyphosate or R1 seed collected, planted, then these plants treated with glyphosate.

Those skilled in the art of corn cell transformation methods can modify this method to provide transgenic corn plants containing a chimeric DNA molecule of the present invention, or use other methods, such as, particle gun, that are known to provide transgenic monocot plants.

### Example 5

### Transgenic Plant Tolerance to Glyphosate

Transgenic Arabidopsis plant that are transformed with the DNA constructs, pMON81517 and pMON81523, and transgenic tobacco plant that are transformed with DNA constructs pMON81517, pMON81523 and pMON81524 were treated with an effective dose of glyphosate sufficient to demonstrate vegetative tolerance and reproductive tolerance. The plants are tested in a greenhouse spray test using Roundup Ultra™ a glyphosate formulation with a Track Sprayer device (Roundup Ultra™ is a registered trademark of Monsanto Company). Plants are treated at the “two” true leaf or greater stage of growth and the leaves are dry before applying the Roundup® spray. The formulation used is Roundup Ultra™ as a 3 lb/gallon a.e. (acid equivalent) formulation. The calibration used is as follows:

For a 20 gallons/Acre spray volume:

| | |
|---|---|
| Nozzle speed: | 9501 evenflow |
| Spray pressure: | 40 psi (pounds per square inch) |
| Spray height | 18 inches between top of canopy and nozzle tip |
| Track Speed | 1.1 ft/sec., corresponding to a reading of 1950 - 1.0 volts. |
| Formulation: | Roundup Ultra ™ (3 lbs. acid equivalent./gallon) |

The spray concentrations will vary, depending on the desired testing ranges. For example, for a desired rate of 8 oz/acre a working solution of 3.1 ml/L is used, and for a desired rate of 64 oz/A a working range of 24.8 ml/L is used. The Arabidopsis plants were treated by spray application of glyphosate at 24 oz/A rate, then evaluated for vegetative tolerance to glyphosate injury and for reproductive tolerance, the results are shown in Table 4. These results show the tolerance to glyphosate in Arabidopsis transformed with two different EPSPS genes, Agrobacterium strain CP4 EPSPS (pMON81517) and Caulobacter crescentus EPSPS-At (pMON81523, contains artificial version of Cc EPSPS with dicot codon bias). A large number to transgenic plant were produced that were determined to be vegetatively tolerant to glyphosate (#Veg tolerant Plants). The glyphosate treated and untreated plants were allowed to flower and set seed. The presence of seed indicated that the plants were fertile. A similar result was observed for the fertility score for the transgenic plants containing pMON81517 (61%) and the pMON81523 (56%) as shown in Table 4. These results indicate that the chimeric DNA molecule containing the coding sequence for the Cc EPSPS provides glyphosate tolerance to transgenic plants at about the same level as the commercial CP4 EPSPS gene. Table 5 shows the reproductive tolerance (% Fertile plants) in tobacco plants transgenic for pMON81517 (CP4 EPSPS), pMON81523 (CcEPSPS artificial), and pMON81524 (CcEPSPS native) treated at 24 oz/A and 96 oz/A. The vegetative glyphosate tolerance of the transgenic tobacco plants from each construct was more then 90% at both rates. At 96 oz/A, the reproductive tolerance shows that the artificial DNA molecule encoding the CcEPSPS (pMON81523) that was modified for enhanced expression provided improved reproductive tolerance relative to the native DNA molecule (pMON81524). The reproductive tolerance was similar to that observed with the commercial standard (CP4 EPSPS). This example provides evidence that modification of the DNA molecules encoding the glyphosate resistant EPSPS enzymes (Table 1) can provide improvement in the glyphosate tolerance observed in transgenic plants containing them.

TABLE 4
Tolerance to glyphosate in transgenic Arabidopsis
Glyphosate treatment 24 oz/A

| | | | #Sterile | |
|---|---|---|---|---|
| Construct | #Veg tolerant plants | #Fertile plants | plants | % Fertile |
| PMON81517 | 62 | 38 | 24 | 61% |
| PMON81523 | 61 | 34 | 27 | 56% |

Untreated controls

| | | | Sterile | |
|---|---|---|---|---|
| Construct | # plants | Fertile plants | plants* | % Fertile |
| PMON81517 | 19 | 13 | 6 | 68% |
| PMON81523 | 28 | 22 | 6 | 79% |

*This group contains plants delayed in development and were classified as sterile.

TABLE 5
Fertility of transgenic tobacco plants
as indication of glyphosate tolerance

| Construct | % Fertile plants 24 oz/A | % Fertile plants 96 oz/A |
|---|---|---|
| PMON81517 | 38 | 23 |
| PMON81523 | 34 | 20 |
| PMON81524 | 37 | 0 |

Corn plants transformed with the DNA constructs of the present invention were observed to be tolerant glyphosate treatment, in particular the DNA constructs pMON81568 and pMON81575 demonstrated a high percentage of glyphosate tolerant plants from those that were transformed. Transformation of corn cells with pMON81568 resulted in a thirty-three percent transformation efficiency and sixty percent of the transgenic plants were tolerant to glyphosate application. Transformation of corn cells with pMON81575 resulted in a thirteen percent transformation efficiency and thirty-six percent of the transgenic plants were tolerant to glyphosate application.

### Example 6

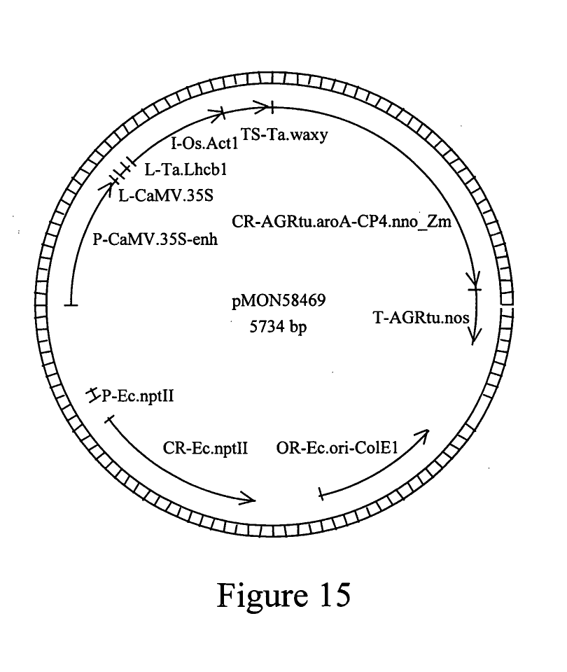

It has been observed that chloroplast transit peptides do not always process precisely, sometimes cleaving in the connected polypeptide and sometimes cleaving in the CTP polypeptide. The result is a processed polypeptide that has variable N-termini. Experiments were conducted to test various CTPs for their ability to process precisely at the junction of the CTP and a glyphosate resistant EPSPS, for example, the CP4 EPSPS. New DNA constructs were created that utilized a wheat GBSS CTP (TS-Ta.Wxy, SEQ ID NO: 38, and CTP-CP4 EPSPS polypeptide SEQ ID NO: 39, FIG. 15 pMON58469), a corn starch branching enzyme II CTP (Zm CsbII, pMON66353, Genbank L08065), a rice soluble starch synthase CTP (Os.Sss, pMON66354, Genbank D16202), a rice EPSPS CTP (Os.EPSPS, pMON66355), a rice GBSS CTP (Os.GBSS, pMON66356, Genbank X62134), a rice tryptophan synthase CTP (Os.trypB, pMON66357, Genbank AB003491), and a corn rubisco CTP (Zm.RbcS2 CTP, pMON58422) fused to the CP4 EPSPS coding sequence to create a chimeric polypeptide. The DNA constructs containing the chimeric CTP-CP4 EPSPS DNA coding sequences were tested for processing in corn protoplasts. Purified plasmid DNA of each DNA construct was introduced into corn leaf protoplast cell by electroporation. The cells were collected and the total protein extracted. The protein extract was separated on a polyacrylamide gel and subjected to western blot analysis (Sambrook et al., 1989) using anti-CP4 EPSPS antibodies. The results indicated that several of the CTP-CP4 EPSPS fusion polypeptides produced multiple processed protein products. The Zm.CsbII CTP-CP4 EPSPS, Os.Sss CTP-CP4 EPSPS, Zm.RbCS2 CTP-CP4 EPSPS, and the Os.TrypB CTP-CP4 EPSPS in particular were observed to produce these products in corn protoplast cells.

The DNA constructs were transformed into rice cells by particle gun (for example, by the methods provided in U.S. Pat. Nos. 6,365,807 and 6,288,312) and the cells regenerated into plants. Analysis of the leaf and seed tissue indicated that the rice EPSPS CTP also produced multiple protein products in rice seed tissue. The wheat GBSS CTP-CP4 EPSPS protein product was purified from transgenic rice seeds and the N-terminus sequence was determined, also the Arabidopsis EPSPS CTP2-CP4 EPSPS DNA construct (pMON32525) was transformed into rice and its protein product purified from rice seed and N-terminus sequenced. The results shown in Table 6 indicate that a single precisely processed mature EPSPS was found when the wheat GBSS CTP was fused to the EPSPS polypeptide. The Arabidopsis CTP was found to produce at least three protein products, one that is correctly processed, one of which has been processed where two amino acids have been removed from the mature EPSPS and one that has been processed with an additional amino acid derived from the CTP. Of the CTP-EPSPS fusion peptides tested, only the wheat GBSS CTP provided precise processing of the mature EPSPS. Additional chimeric DNA molecules were created that encode the wheat GBSS CTP fused to the Xc EPSPS (SEQ ID NO: 40) and to the Cc EPSPS (SEQ ID NO: 41). The wheat GBSS CTP can be fused to any EPSPS to enhance precise processing to the mature EPSPS. In particular, the CP4 EPSPS and EPSPS enzymes derived from Table 1. Also, other agronomically useful proteins can be fused with the wheat GBSS CTP for use as a transgene to provide novel phenotypes to crop plants.

TABLE 6
Analysis of the N-terminus of transgenic
plant produced CTP-EPSPS

| | |
|---|---|
| Mature CP4 EPSPS | MLHGAXSRXATA . . . |
| Wheat GBSS CTP-CP4 EPSPS | MLHGAXSRXATA . . . |
| Arabidopsis CTP-CP4 EPSPS | MLHGAXSRXATA . . . |
| | GASSRPATA . . . |
| | XMLHGASXRPAT . . . |

Having illustrated and described the principles of the present invention, it should be apparent to persons skilled in the art that the invention can be modified in arrangement and detail without departing from such principles. We claim all modifications that are within the spirit and scope of the appended claims.

All publications and published patent documents cited in this specification are incorporated herein by reference to the same extent as if each individual publication or patent application was specifically and individually indicated to be incorporated by reference.

1. A chimeric DNA molecule comprising a promoter molecule functional in a plant cell operably connected to a polynucleotide molecule encoding a glyphosate resistant 5-enolpyruvyl-3-phosphoshikimate synthase polypeptide, wherein said 5-enolpyruvyl-3-phosphoshikimate synthase polypeptide comprises the sequence domains X₁-D-K-S, in which X₁ is G or A or S or P; S-A-Q-X₂-K, in which X₂ is any amino acid; and R-X₃-X₄-X₅-X₆, in which X₃ is D or N, X₄ is Y or H, X₅ is T or S, X₆ is R or E; and N-X₇-X₈-R, in which X₇ is P or E or Q, and X₈ is R or L.

2. The chimeric DNA molecule of claim 1, wherein said 5-enolpyruvyl-3-phosphoshikimate synthase polypeptide comprises the sequence domains X₁-D-K-S, in which X₁ is G; S-A-Q-X₂-K, in which X₂ is I or V; and R-X₃-X₄-X₅-X₆, in which X₃ is D or N, X₄ is Y or H, X₅ is T, X₆ is R or E; and N-X₇-X₈-R, in which X₇ is P or E or Q, and X₈ is R or L.

3. The chimeric DNA molecule of claim 1, wherein said 5-enolpyruvyl-3-phosphoshikimate synthase polypeptide comprises the sequence domains X₁-D-K-S, in which X₁ is G; S-A-Q-X₂-K, in which X₂ is I or V; and R-X₃-X₄-X₅-X₆, in which X₃ is D, X₄ is H, X₅ is T, X₆ is E; and N-X₇-X₈-R, in which X₇ is P or E, and X₈ is L.

4. The DNA molecule of claim 1, wherein said 5-enolpyruvyl-3-phosphoshikimate synthase polypeptide comprises the sequence domains X₁-D-K-S, in which X₁ is A or S or P; S-A-Q-X₂-K, in which X₂ is V; and R-X₃-X₄-X₅-X₆, in which X₃ is D or N, X₄ is H, X₅ is T or S, X₆ is E; and N-X₇-X₈-R, in which X₇ is P or Q, and X₈ is R.

5. The chimeric DNA molecule of claim 1, wherein the polynucleotide molecule encodes a 5-enolpyruvyl-3-phosphoshikimate synthase polypeptide, the polypeptide selected from the group consisting of SEQ ID NO: 5-18.

6. The chimeric DNA molecule of claim 1, wherein the polynucleotide molecule encodes a glyphosate resistant 5-enolpyruvyl-3-phosphoshikimate synthase polypeptide, the polynucleotide selected from the group consisting of SEQ ID NO: 19-32.

7. The chimeric DNA molecule of claim 1, wherein the promoter is selected from the group consisting of the rice actin 1 promoter, rice tubulin A promoter, Arabidopsis actin 7 promoter, CaMV 35S promoter, FMV promoter, elongation factor 1 alpha promoter, chimeric fusion of the FMV promoter and elongation factor 1 alpha promoter, and chimeric fusion of the CaMV 35S promoter and actin 8 promoter.

8. The chimeric DNA molecule of claim 1, wherein the polynucleotide molecule encodes a glyphosate resistant 5-enolpyruvyl-3-phosphoshikimate synthase, the polynucleotide comprising modifications for enhanced expression in plant cells.

9. The chimeric DNA molecule of claim 8, wherein said polynucleotide molecule is selected from the group consisting of SEQ ID NO: 33-37.

10. The chimeric DNA molecule of claim 1, wherein said molecule is contained within the germplasm of a plant.

11. The chimeric DNA molecule of claim 10, wherein said plant is a monocot plant and is tolerant to glyphosate herbicide relative to a non-transformed monocot plant of the same species.

12. The chimeric DNA molecule of claim 10, wherein said plant is a dicot plant and is tolerant to glyphosate herbicide relative to a non-transformed dicot plant of the same species.

13. The chimeric DNA molecule of claim 10, wherein said molecule is contained within a material processed from said germplasm of a plant.

14. The chimeric DNA molecule of claim 1 further comprising a second polynucleic acid molecule encoding a chloroplast transit peptide operably linked with, and in the order of transcription between, the promoter functional in a plant cell and the polynucleotide molecule encoding a glyphosate resistant 5-enolpyruvyl-3-phosphoshikimate synthase polypeptide.

15. A chimeric DNA molecule comprising a promoter molecule functional in a plant cell operably connected to a polynucleotide molecule encoding a glyphosate resistant 5-enolpyruvyl-3-phosphoshikimate synthase polypeptide, wherein said polypeptide comprises the sequence domain S-A-Q-X₂-K, in which X₂ is any amino acid; and does not contain the sequence domains -G-D-K-X₃- in which X₃ is Ser or Thr, and R-X₁-H-X₂-E- in which X₁ is an uncharged polar or acidic amino acid and X₂ is Ser or Thr, and -N-X₅-T-R- in which X₅ is any amino acid.

16. The chimeric DNA molecule of claim 15, wherein said molecule is contained within the germplasm of a plant.

17. The chimeric DNA molecule of claim 16, wherein said plant is a monocot plant and is tolerant to glyphosate herbicide relative to a non-transformed monocot plant of the same species.

18. The chimeric DNA molecule of claim 16, wherein said plant is a dicot plant and is tolerant to glyphosate herbicide relative to a non-transformed dicot plant of the same species.

19. The chimeric DNA molecule of claim 16, wherein said molecule is contained within a material processed from said germplasm of a plant.

20. A chimeric DNA molecule comprising a first polynucleotide molecule of a promoter functional in a plant cell operably linked to a second polynucleotide encoding a wheat Granule bound starch synthase chloroplast transit peptide operably linked with a third heterologous polynucleotide molecule that encodes a polypeptide to be transported to a plant chloroplast.

21. The chimeric DNA molecule of claim 20, wherein said second polynucleotide molecule encodes a chloroplast transit peptide consisting essentially of SEQ ID NO: 38.

22. The chimeric DNA molecule of claim 20, wherein said third polynucleotide encodes for a glyphosate resistant 5-enolpyruvyl-3-phosphoshikimate synthase polypeptide.

23. The chimeric DNA molecule of claim 20, wherein said second polynucleotide and said third polynucleotide form a chimeric polynucleotide molecule selected from the group consisting of SEQ ID NO: 39-41.

24. The chimeric DNA molecule of claim 20, wherein said molecule is contained within the germplasm of a plant.

25. The chimeric DNA molecule of claim 24, wherein said plant is a monocot plant.

26. The chimeric DNA molecule of claim 20, wherein said plant is a dicot plant.

27. The chimeric DNA molecule of claim 24, wherein said molecule is contained within a material processed from said germplasm of a plant.

28. A method for selectively killing weeds in a field of crop plants, the method comprising the steps of: a) planting crop seeds or plants that have glyphosate tolerance as a result of a chimeric DNA molecule being inserted into the genome of said crop seeds or plants, said DNA molecule comprising the DNA molecule of claim 1 or claim 15; and b) applying to said crop seeds or plants a sufficient amount of glyphosate that inhibits the growth of glyphosate sensitive plants, wherein said amount of glyphosate does not significantly affect said crop seeds or plants that comprise the chimeric DNA molecule.


SEQUENCE LISTING (51 sequences)

SEQ ID NO: 1 - protein, 4 aa, Artificial Sequence
  motif 1 of an EPSPS protein
XDKS

SEQ ID NO: 2 - protein, 5 aa, Artificial Sequence
  motif 2 of an EPSPS
SAQXK

SEQ ID NO: 3 - protein, 5 aa, Artificial Sequence
  motif 3 of an EPSPS
RXXXX

SEQ ID NO: 4 - protein, 4 aa, Artificial Sequence
  motif 4 of an EPSPS
NXXR

SEQ ID NO: 5 - protein, 442 aa, Xylella fastidiosa
MSHRTHDYWIAHQGTPLHGVLSIPGDKSISHRAVMFAALADGTSRIDGFLEAEDTCSTAEILARLGVRIETPLSTQRIVHGVGVDGLQASHIPLDCGNAGTGMRLLAGLLVAQPFDSVLVGDASLSKRPMRRVTDPLSQMGARIDTSDDGTPPLRIYGGQLLHGIDFISPVASAQIKSAVLLAGLYARNETVVREPHPTRDYTERMLTAFGVDIDVSTGCARLRGGQRLCATDITIPADFSSAAFYLVAASVIPGSDITLRAVGLNPRRIGLLTVLRLMGANIVESNRHEQGGEPVVDLRVRYAPLQGTRVPEDLVADMIDEFPALFVAAAAAEGQTVVSGAAELRVKESDRLAAMVTGLRVLGVQVDETADGATIHGGPIGHGTINSHGDHRIAMAFSIAGQLSVSTVRIEDVANVATSFPDYETLARSAGFGLEVYCDPA

SEQ ID NO: 6 - protein, 438 aa, Xanthomonas campestris
MSNSSQHWIAQRGTALQGSLTIPGDKSVSHRAVMFAALADGTSKIDGFLEGEDTRSTAAIFAQLGVRIETPSASQRIVHGVGVDGLQPPQGPLDCGNAGTGMRLLAGVLAAQRFDSVLVGDASLSKRPMRRVTGPLAQMGARIETESDGTPPLRVHGGQPLQGITFASPVASAQVKSAVLLAGLYAAGETSVSEPHPTRDYTERMLSAFGVDIAFSPGQARLRGGQRLRATDIAVPADFSSAAFFIVAASIIPGSDVTLRAVGLNPRRTGLLAALRLMGADIVEDNHAEHGGEPVADLRVRYAPLQGAQIPEALVPDMIDEFPALFVAAAAARGDTVVSGAAELRVKESDRLAAMATGLRALGIVVDETPDGATIHGGTLGSGVIESHGDHRIAMAFAIAGQLSTGTVQVNDVANVATSFPGFDSLAQGAGFGLSARP

SEQ ID NO: 7 - protein, 467 aa, Rhodopseudomonas palustris
MPKAARRRDARPNHPQPRGTTILTDSNQPMPLQARKSGALHGTARVPGDKSISHRALILGALAVGETRISGLLEGEDVINTAKAMRALGAKVERTGDCEWRVHGVGVAGFATPEAPLDFGNSGTGCRLAMGAVAGSPIVATFDGDASLRSRPMRRIVDPLELMGAKVVSSSEGGRLPLALQGARDPLPILYRTPVPSAQIKSAVLLAGLSAPGITTVIEAEASRDHTELMLQHFGATIVTEAEGAHGRKISLTGQPELRGAPVVVPADPSSAAFPMVAALVVPGSDIELTDVMTNPLRTGLITTLREMGASIEDSDVRGDAGEPMARFRVRGSKLKGVEVPPERAPSMIDEYLVLAVAAAFAEGTTVMRGLHELRVKESDRLEATAAMLRVNGVAVEIAGDDLIVEGKGHVPGGGVVATHMDHRIAMSALAMGLASDKPVTVDDTAFIATSFPDFVPMMQRLGAEFG

SEQ ID NO: 8 - protein, 488 aa, Magnetospirillum magnetotacticum
MFPTLCQNEKAWAVQHGTQVYDAKGACDRASAGSFLPCRWLSGVIMAKPLSSRKAAPLAGSARVPGDKSISHRALMLGALAVGESVVTGLLEGDDVLRTAACMRALGAEVERQADGSWRLFGRGVGGLMEPADILDMGNSGTGARLLMGLVATHPFTCFFTGDGSLRSRPMRRVIEPLSRMGARFVSRDGGRLPLAVTGTSQPTPITYELPVASAQVKSAIMLAGLNTAGETTVIEREATRDHTELMLRNFGATVRVEDAEGGGRAVTVVGFPELTGRPVVVPADPSSAAFPVVAALLVEGSEIRLPGVGTNPLRTGLYQTLLEMGADIRFDNPRDQAGEPVADLVVRASRLKGVDVPAERAPSMIDEYPILAVAAAFAEGTTRMRGLAELRVKESDRLAAMARGLAACGVAVEEEKDSLIVHGTGRIPDGDATVTTHFDHRIAMSFLVMGMASARPVAVDDAEAIETSFPIFVELMNGLGAKIEAMG

SEQ ID NO: 9 - protein, 443 aa, Caulobacter crescentus
MSLAGLKSAPGGALRGIVRAPGDKSISHRSMILGALATGTTTVEGLLEGDDVLATARAMQAFGARIEREGVGRWRIEGKGGFEEPVDVIDCGNAGTGVRLIMGAAAGFAMCATFTGDQSLRGRPMGRVLDPLARMGATWLGRDKGRLPLTLKGGNLRGLNYTLPMASAQVKSAVLLAGLHAEGGVEVIEPEATRDHTERMLRAFGAEVIVEDRKAGDKTFRHVRLPEGQKLTGTHVAVPGDPSSAAFPLVAALIVPGSEVTVEGVMLNELRTGLFTTLQEMGADLVISNVRVASGEEVGDITARYSQLKGVVVPPERAPSMIDEYPILAVAAAFASGETVMRGVGEMRVKESDRISLTANGLKACGVQVVEEPEGFIVTGTGQPPKGGATVVTHGDHRIAMSHLILGMAAQAEVAVDEPGMIATSFPGFADLMRGLGATLAEA

SEQ ID NO: 10 - protein, 445 aa, Magnetococcus sp. MC-1
MSSTHPGRTIRSGATQNLSGTIRPAADKSISHRSVIFGALAEGETHVKGMLEGEDVLRTITAFRTMGISIERCNEGEYRIQGQGLDGLKEPDDVLDMGNSGTAMRLLCGLLASQPFHSILTGDHSLRSRPMGRVVQPLTKMGARIRGRDGGRLAPLAIEGTELVPITYNSPIASAQVKSAIILAGLNTAGETTIIEPAVSRDHTERMLIAFGAEVTRDGNQVTIEGWPNLQGQEIEVPADISAAAFPMVAALITPGSDIILENVGMNPTRTGILDLLLAMGGNIQRLNEREVGGEPVADLQVRYSQLQGIEIDPTVVPRAIDEFPVFFVAAALAQGQTLVQGAEELRVKESDRITAMANGLKALGAIIEERPDGALITGNPDGLAGGASVDSFTDHRIAMSLLVAGLRCKESVLVQRCDNINTSFPSFSQLMNSLGFQLEDVSHG

SEQ ID NO: 11 - protein, 428 aa, Enterococcus faecalis
MRVQLRTNVKHLQGTLMVPSDKSISHRSIMFGAISSGKTTITNFLRGEDCLSTLAAFRSLGVNIEDDGTTITVEGRGFAGLKKAKNTIDVGNSGTTIRLMLGILAGCPFETRLAGDASIAKRPMNRVMLPLNQMGAECQGVQQTEFPPISIRGTQNLQPIDYTMPVASAQVKSAILFAALQAEGTSVVVEKEKTRDHTEEMIRQFGGTLEVDGKKIMLTGPQQLTGQNVVVPGDISSAAFFLVAGLVVPDSEILLKNVGLNQTRTGILDVIKNMGGSVTILNEDEANHSGDLLVKTSQLTATEIGGAIIPRLIDELPIIALLATQATGTTIIRDAEELKVKETNRIDAVAKELTILGADITPTDDGLIIHGPTSLHGGRVTSYGDHRIGMMLQIAALLVKEGTVELDKAEAVSVSYPAFFDDLERLSC

SEQ ID NO: 12 - protein, 428 aa, Enterococcus faecalis
MRVQLRTNVKHLQGTLMVPSDKSISHRSIMFGAISSGKTTITNFLRGEDCLSTLAAFRSLGVNIEDVGTTITVEGQGFAGLKKAKNTIDVGNSGTTIRLMLGILAGCPFETRLAGDASISKRPMNRVMLPLNQMGAECQGVQQTEFPPISIRGTQNLQPIDYTMPVASAQVKSAILFAALQAEGTSVVVEKEKTRDHTEEMIRQFGGTLEVDGKKIMLTGPQQLTGQNVVVPGDISSAAFFLVAGLVVPDSEILLKNVGLNQTRTGILDVIKNMGGSVTILNEDEANHSGDLLVKTSQLTATEIGGAIIPRLIDELPIIALLATQATGTTIIRDAEELKVKETNRIDAVAKELTILGADITPTDDGLIIHGPTSLHGGRVTSYGDHRIGMMLQIAALLVKEGTVELDKAEAVSVSYPAFFDDLERLSC

SEQ ID NO: 13 - protein, 289 aa, Enterococcus faecium
MRLLQQIHGLRGTVRIPADKSISHRSIMFGAIAEGTTTIQNFLRAEDCLSTLHAFQQLGVEIEEEEEVIKIHGRGSHSFVQPTAPIDMGNSGTTSRLLMGILAGQPFTTTLVGDASLSKRPMGRVMEPLREMGADLQGNESDQYLPITVTGTRSLSTIRYNMPVASAQVKSALLFAALQAEGTSVIVEKERSRNHTEEMIRQFGGRITVEDKTIMVTGPQKLTGQQITVPGDISSAAFFLAAGLLVPESQLLLKNVGVNPTRTGILDVLEEMGARLPRRITMNITNRLI

SEQ ID NO: 14 - protein, 354 aa, Thermotoga maritima
MKVFPKPFAEPIEPLFCGNSGTTTRLMSGVLASYEMFTVLYGDPSLSRRPMRRVIEPLEMMGARFMARQNNYLPMAIKGNHLSGISYKTPVASAQVKSAVLLAGLRASGRTIVIEPAKSRDHTERMLKNLGVPVEVEGTRVVLEPATFRGFTMKVPGDISSAAFFVVLGAIHPNARITVTDVGLNPTRTGLLEVMKLMGANLEWEITEENLEPIGTVRVETSPNLKGVVVPEHLVPLMIDELPLVALLGVFAEGETVVRNAEELRKKESDRIRVLVENFKRLGVEIEEFKDGFKIVGKQSIKGGSVDPEGDHRMAMLFSIAGLVSEEGVDVKDHECVAVSFPNFYELLERVVIS

SEQ ID NO: 15 - protein, 431 aa, Aquifex aeolicus
MKKIEKIKRVKGELRVPSDKSITHRAFILGALASGETLVRKPLISGDTLATLEILKAIRTKVREGKEEVLIEGRNYTFLEPHDVLDAKNSGTTARIMSGVLSTQPFFSVLTGDESLKNRPMLRVVEPLREMGAKIDGREEGNKLPIAIRGGNLKGISYFNKKSSAQVKSALLLAGLRAEGMTEVVEPYLSRDHTERMLKLFGAEVITIPEERGHIVKIKGGQELQGTEVYCPADPSSAAYFAALATLAPEGEIRLKEVLLNPTRDGFYRKLIEMGGDISFENYRELSNEPMADLVVRPVDNLKPVKVSPEEVPTLIDEIPILAVLMAFADGVSEVKGAKELRYKESDRIKAIVTNLRKLGVQVEEFEDGFAIHGTKEIKGGVIETFKDHRIAMAFAVLGLVVEEEVIIDHPECVTVSYPEFWEDILKVVEF

SEQ ID NO: 16 - protein, 395 aa, Helicobacter pylori
MGEDCLSSLEIAQNLGAKVENTAKNSFKITPPTTIKEPNKILNCNNSGTSMRLYSGLLSAQKGLFVLSGDNSLNARPMKRIIEPLKAFGAKILGREDNHFAPLAIVGGPLKACDYESPIASAQVKSAFILSALQAQGISAYKESELSRNHTEIMLKSLGANIQNQDGVLKISPLEKPLESFDFTIANDPSSAFFLALACAITPKSRLLLKNVLLNPTRIEAFEVLKKMGAHIEYVIQSKDLEVIGDIYIEHAPLKAISIDQNIASLIDEIPALSIAMLFAKGKSMVRNAKDLRAKESDRIKAVVSNFKALGIECEEFEDGFYIEGLGDASQLKQHFSKIKPPIIKSFNDHRIAMSFAVLTLALPLEIDNLECANISFPTFQLWLNLFKKRSLNGN

SEQ ID NO: 17 - protein, 395 aa, Helicobacter pylori
MGEDCLSSLEIAQNLGAKVENTAKNSFKITPPTTIKEPNKILNCNNSGTTMRLYSGLLSAQKGLFVLSGDNSLNARPMKRIIEPLKAFGAKILGREDNHFAPLVILGSPLKACHYESPIASAQVKSAFILSALQAQGASTYKESELSRNHTEIMLKSLGADIHNQDGVLKISPLEKPLEAFDFTIANDPSSAFFFALACAITPKSRLLLKNVLLNPTRIEAFEVLKKMGASIEYAIQSKDLEMIGDIYVEHAPLKAINIDQNIASLIDEIPALSIAMLFAKGKSMVKNAKDLRAKESDRIKAVVSNFKALGIECEEFEDGFYVEGLEDISPLKQRFSRIKPPLIKSFNDHRIAMSFAVLTLALPLEIDNLECANISFPQFKHLLNQFKKGSLNGN

SEQ ID NO: 18 - protein, 428 aa, Campylobacter jejuni
MKIYKLQTPVNAILENIAADKSISHRFAIFSLLTQEENKAQNYLLAQDTLNTLEIIKNLGAKIEQKDSCVKIIPPKEILSPNCILDCGNSGTAMRLMIGFLAGISGFFVLSGDKYLNNRPMRRISKPLTQIGARIYGRNEANLAPLCIEGQKLKAFNFKSEISSAQVKTAMILSAFRADNVCTFSEISLSRNHSENMLKAMKAPIRVSNDGLSLEINPLKKPLKAQNIIIPNDPSSAFYFVLAAIILPKSQIILKNILLNPTRIEAYKILQKMGAKLEMTITQNDFETIGEIRVESSKLNGIEVKDNIAWLIDEAPALAIAFALAKGKSSLINAKELRVKESDRIAVMVENLKLCGVEARELDDGFEIEGGCELKSSKIKSYGDHRIAMSFAILGLLCGIEIDDSDCIKTSFPNFIEILSNLGARIDY

SEQ ID NO: 19 - DNA, 1329 bp, Xylella fastidiosa
atgagtcatagaacgcatgactattggatcgcacaccagggcaccccactgcatggtgtcctgagtatccccggcgataaatcaatctcccatcgtgcagtcatgtttgctgcgcttgcggatggcacgtcacgtattgatggctttcttgaggcggaggatacgtgctctacagcagagatcttggcccgattgggtgtgcgtatcgaaactcccttatccacgcagcgcatcgtccatggtgttggtgtggatggacttcaggcatcgcatattcccctggattgtggcaatgcaggcactggcatgcgcctgctcgctggtttgctggtagcgcagccttttgacagcgtcttagtcggagatgcatcactgtccaagcgaccgatgcgacgtgtgacggatccgctgtcacagatgggcgcacgtatcgataccagtgacgatggcactccaccgctgcgtatttacggtggtcaattactccacggtatcgattttatctccccagtggccagtgctcagatcaagtcagcggtgttgctggctggattgtatgcacgtaacgaaacggtagtgcgtgaaccgcacccgacgcgtgattacaccgagcgtatgctcactgcgtttggtgtggacattgatgtttccacagggtgcgcgcgcttgcgtggtgggcaacggttatgtgctaccgatattacaatcccggctgatttttcctcagctgcgttttatctggttgcagccagcgtgattcctggctctgatatcaccctgcgtgctgttggactcaatccgcgtcgtattggtttgttaaccgtgttgcggctgatgggggcaaatattgttgaatccaatcgccatgaacagggtggtgagccggttgttgacctacgtgtgcgttatgcgccactccagggcacccgtgttcctgaagatttggtggcggatatgattgacgaattcccggccttgtttgtcgctgcagcggcagccgaaggtcaaacggtagtgagtggtgcggctgaactacgcgttaaagaatcggaccggttggctgcgatggtgacaggcttgcgcgtgcttggcgttcaggtggatgagaccgccgacggggcaacgattcatggagggcccatcggtcatggcaccatcaacagccatggcgatcaccgcatcgccatggcgttttcaattgcaggtcagctttctgtcagtacagtacgtattgaagatgtcgccaatgttgcgacttcttttccagactatgagacgttagcgcgcagcgctggtttcggtcttgaggtgtactgcgatccagcatga

SEQ ID NO: 20 - DNA, 1317 bp, Xanthomonas campestris
atgagcaacagctcgcaacactggatcgcacagcgcggcaccgcgctgcagggcagcctgaccattcccggcgacaagtcggtttcgcaccgcgcggtgatgttcgccgcactggcggatggcacctcaaagatcgacggctttctggaaggcgaagacacgcgttccaccgcggcgatctttgcccagctgggcgtgcgcattgaaacgccgtcggcgtcgcagcgcatcgtgcatggcgtcggtgtggacggcctacagccgccgcaggggccgctggattgtggcaacgccggcaccggcatgcgcttgctggccggcgtgctcgcggcgcagcggttcgatagcgtactggtgggcgatgcgtcgttgtccaagcggcccatgcgccgcgtcaccggcccgctggcgcagatgggtgcacgcatcgaaaccgaatcggatggcacgccgccgctgcgtgtccacggcggccagccgctgcaaggcattacgtttgcctcgccggtggctagtgcgcaggtcaaatcggccgtgctgctggccgggttgtacgcagcgggtgagacctcggtgagtgagccgcatcctacgcgcgactacaccgaacgcatgctctccgcattcggcgtggacatcgcgttttctcctggccaggcgcgtctgcgtggcggccagcgtttgcgtgcgaccgatatcgcggtgccggcagatttttcatcggcggcgttcttcatcgtggccgccagcatcattcccggctcggacgtgactttgcgtgcggtaggtctgaatccgcggcgcaccggccttttggccgccctgcggctgatgggcgccgatatcgtggaagacaatcacgccgaacacggcggtgagccggtggcggacctgcgcgtgcgctacgcaccgctgcagggcgcgcagattcccgaagcgctggtgccggacatgatcgatgagttcccggcgctattcgtcgccgcagctgcggcgcgcggcgacacggtcgtcagtggtgcggcggaattgcgcgtcaaggaatccgatcgtctcgccgcgatggccaccggcctgcgggcgctcggcattgtggtggacgaaacgccggacggtgccaccattcacggcggcacgctgggcagcggcgtcatcgaaagccacggcgatcaccgcattgcaatggcgtttgccatcgcaggccagctgtcgaccgggacggtacaggtcaacgacgtggcgaacgtggccacctcgttcccaggcttcgacagcctggcgcagggcgccgggttcgggctcagcgcgcgtccgtga

SEQ ID NO: 21 - DNA, 1404 bp, Rhodopseudomonas palustris
atgccgaaggccgcgaggcgccgcgacgccaggccgaatcacccgcagccccgagggaccaccatcttgactgattcgaaccagccgatgccgctgcaggcgcgcaagagcggcgcattgcatggcaccgcgcgcgtcccaggcgacaagtcgatttcgcaccgggcgctgattctcggcgcgctggcggtcggcgagacccgaatctccggcttgctcgagggcgaagacgtcatcaacaccgccaaagcgatgcgcgcgctcggtgccaaggtcgagcgcaccggcgactgcgaatggcgcgtgcatggcgtcggcgttgcaggctttgcgacgccggaggccccgctggatttcggcaattcgggcaccggctgccgtttggcgatgggcgcggtggccggatcgcctattgtggcgaccttcgacggcgatgcatcgctgcgcagccggccgatgcggcgaatcgtcgatcccttggagctgatgggtgccaaggtggtgtcgagcagcgagggcggccgattgccgctggccctacagggcgcccgcgatccgctgccgattctgtaccgcaccccggtgccgtcggcgcagatcaaatccgccgtgctgctcgccggcctgtcggcgcccggcatcactaccgtgatcgaggccgaggccagccgcgaccataccgagctgatgctgcagcatttcggcgccacgatcgtcaccgaagccgaaggtgcccatggccgtaagatttcattaaccggccagcccgaattgcgcggcgccccggtggtggtgccggccgatccgtcttcggcggcctttccgatggtcgcggcgctggtggtgcccggctccgatatcgaattgaccgacgtgatgaccaacccgctgcgcaccgggttgatcacgacgctgcgcgaaatgggcgcctcgatcgaggacagcgacgtccggggcgatgccggcgagccgatggcccggttccgggtgcgcggttcgaagctgaagggcgtcgaggtgccgccggaacgcgcgccgtcgatgatcgacgagtatctggtgctggcggtcgccgctgcgttcgccgaaggcaccaccgtgatgcgcggcctccacgaactgcgggtcaaggaaagcgaccggctggaagcgacggcggcgatgctgcgggtcaacggcgtcgcggtcgagatcgcaggcgacgatctgatcgtcgagggtaagggccatgtgccgggcggcggtgtggtcgccacccacatggatcatcgcatcgcgatgtcggctctcgccatgggcctcgcctcggacaagccggtgacggtcgacgacaccgccttcatcgccaccagcttcccggacttcgttccgatgatgcagcggctcggcgcggaattcggctga

SEQ ID NO: 22 - DNA, 1466 bp, Magnetospirillum magnetotacticum
atgttccccaccctgtgtcaaaacgaaaaagcgtgggcggtgcagcatggaacgcaggtctatgacgcgaagggcgcctgtgatagagcttcggcgggcagctttctgccttgccgctggttatcaggagtgatcatggccaagccgctttcttcccgtaaggccgcaccgttggccggttcggcgcgagttccgggcgacaaatccatctcgcaccgcgccttgatgctgggcgcgctggcggtgggcgaaagcgtggtgaccggccttttggaaggcgacgatgttttacgcacggctgcctgcatgcgagccttgggggccgaggtggagcgtcaggccgacgggtcgtggcggctgttcggcaggggcgtcggtgggctgatggagccagccgacattctcgacatgggcaattccgggacgggagcgcgcctgctgatggggctggtggcgacccatcccttcacatgtttctttaccggcgatggctcgctgcggtcacggcccatgcgccgggtgatcgagcccctgtcgcgcatgggagcgcgcttcgtcagccgcgacggcgggcgcctgcccctggcggtgaccggcacctcccagcccacccccatcacttacgagcttcccgtggcctcggcccaggtgaagtcggccatcatgctggctggcctcaataccgctggcgagaccacggtgatcgagcgcgaggccacccgtgaccacaccgaactgatgctcaggaatttcggcgctaccgtgcgggtcgaggatgccgaaggcggcggccgggccgtcaccgtggtgggctttcccgaactgaccggccgcccggtggtggtgcccgccgacccgtcctcggccgccttcccggtggtggccgccctgctggtggagggctcggaaatccgcctgcccggcgtgggcaccaatcccttgcgcaccggcctgtaccagaccctgctggaaatgggcgccgatatccgcttcgacaatccccgcgatcaggcgggcgagccggtggccgatctggtggtgcgtgcttcaaggctgaaaggcgtcgacgtccctgccgagcgggcgccctccatgatcgacgaataccccatcctggccgtggccgccgccttcgccgagggcaccacccgcatgcgggggctggccgagcttcgggtcaaggaaagcgaccgcctggccgccatggcgcgcggactggccgcctgcggcgtggcggtggaggaggagaaggattccctcatcgttcacggcacgggacgcattcccgacggcgacgccacggtgaccacccatttcgaccatcgcatcgccatgtccttcctggtcatgggcatggcctcggcccggcccgtggcggtggacgacgccgaagccatcgagaccagcttccccatcttcgtcgaactgatgaatgggttgggggcgaagatcgaggcgatggggtg

SEQ ID NO: 23 - DNA, 1332 bp, Caulobacter crescentus
atgtcgctggctggattgaagagcgctcccggaggcgctctgcgagggatcgtgcgcgctccgggagacaagtccatttctcaccgttcgatgatcctgggcgcgctggcgaccgggacgacgacggtcgaaggtctcctggaaggggacgacgtcctggccaccgcccgggccatgcaggcctttggcgcgcggatcgaacgcgagggcgtcgggcgctggcggatcgagggcaagggcggctttgaagagcccgtcgacgtcatcgactgcggcaacgccggcaccggcgtgcgcctgatcatgggcgcggcggcgggctttgcgatgtgcgccaccttcacgggcgaccagtcgctgcgcggacgcccgatgggccgggtgctggatccgctggcccgcatgggcgcgacctggctgggtcgcgacaagggccgcctgcccttgaccctgaagggcggaaacctgcgcggcctcaactacaccctgcccatggcctcggcccaggtgaagtcggccgtgctgctggcgggcctgcacgccgagggcggcgtcgaggtcatcgagcctgaagccacgcgcgaccacaccgagcggatgctgcgcgccttcggggctgaggtgatcgtcgaggaccgcaaggccggcgacaagaccttccgccatgtgcgcctgcctgaggggcagaaactgaccggaacccacgtggccgtgccgggcgacccctcgtcggccgcgttcccgctggtggcggccctgatcgttcccggctcggaagtgacggtcgagggcgtgatgctcaacgaactgcgcacgggtctcttcaccaccctgcaggagatgggcgcggatctcgtgatctcgaacgtgcgcgtcgccagcggcgaggaggtcggcgacatcaccgcgcgctactcccagctcaagggcgtcgtcgtgccgcccgagcgcgcgccgtcgatgatcgacgagtatccgatcctggccgtggccgcggcttttgcgtccggcgagacggtgatgcgcggcgtcggcgagatgcgcgtcaaggaaagcgaccgcatcagcctgaccgccaatggcctgaaggcgtgcggggtccaggtggtcgaggagcctgaaggcttcatcgtcaccgggaccggccagccgccgaagggcggggcgaccgtcgtcacccacggcgaccaccgcatcgccatgagccacctgatcctgggcatggccgcccaggcggaggtcgccgtcgacgagccgggcatgatcgccaccagcttcccaggcttcgccgacctgatgcgcggcctgggcgcgacgctggcggaggcctga

SEQ ID NO: 24 - DNA, 1338 bp, Magnetococcus sp. MC-1
atgtccagcacccatcccggacgcaccatccgtagcggcgccacgcaaaacctctccggcaccatccgccccgccgccgataaatccatctcccaccgctccgtgatctttggcgccctggccgaaggcgaaacccacgttaaaggcatgctggaaggcgaagatgtgctgcgtaccatcaccgcctttcgtaccatgggtatctctatcgaacgctgcaacgaaggtgaataccgcatccaaggccaaggactcgacggcctaaaagaacccgatgacgtgctggatatgggtaactccggtaccgccatgcgcctgctgtgcggcctgctggccagccaaccctttcactctatcctcaccggcgatcactccctacgcagccgccccatgggccgcgtagtgcaacccctaaccaaaatgggcgctcgcatccgtggccgcgacggtggccgcctggcccccctcgccatcgaaggcactgaactggtacccattacctacaatagccccatcgcctcggcccaagtgaagtccgccattatcctggccggactcaataccgccggcgaaaccaccatcattgaacccgccgtcagccgcgaccacaccgaacgtatgctcatcgccttcggtgccgaagtgacccgcgatggcaaccaagtgaccatcgaaggctggcccaacctgcaaggccaagagatcgaagtgcccgccgatatctccgccgccgccttccccatggtggccgcccttatcaccccaggatctgatattatcctggaaaatgtgggtatgaacccaacccgtaccggtattctcgacctgctcctggctatgggcggcaatatccaacgcctcaacgaacgggaagttggcggcgaacccgtggccgacctacaggtgcgctactcccaactccaaggcatcgagatagaccccaccgtggtgccccgtgccattgatgagttccccgtgttttttgtagccgccgccctcgcccaaggccaaaccctggtgcaaggcgccgaagagctgcgcgttaaagagagcgaccgcatcaccgccatggccaacggtcttaaagccctaggtgccatcatagaagaacgccccgatggcgcacttattaccggaaatcccgacggtctggccggtggggccagcgtagactcctttaccgaccaccgtatcgccatgagcctgctggtggccggcctgcgctgtaaagagtccgtattggtgcaacgctgcgataatatcaatacctcctttcccagcttttcccaattaatgaacagtcttggttttcaattggaggatgtcagccatggctga

SEQ ID NO: 25 - DNA, 1287 bp, Enterococcus faecalis
atgagggtgcaactacgtacaaatgtgaagcatttacaagggactctgatggttcctagcgacaaatcgatttcccatagaagtattatgtttggagcgatttcttctggaaaaacgacgattacaaattttctaagaggcgaagattgtttaagtaccttagcggcgtttcgttctttaggtgtgaacattgaagatgacgggacgacaatcaccgttgaggggcgaggatttgcaggcttaaaaaaggcgaagaatacaattgatgttggaaattcagggacaacaattcgtctgatgctgggcattttagctggctgtccctttgaaacgcgcctagctggtgatgcgtctattgccaaacgaccaatgaatcgtgtaatgcttcctttaaaccaaatgggagcggaatgtcaaggggttcagcaaacggagtttccgccaatttctattcgcgggactcaaaatttgcaaccgattgactacacaatgcctgttgcaagtgctcaagttaaatcggctattttattcgccgctttgcaagccgagggcacttctgtagtggttgagaaagaaaagacacgtgatcatacagaagagatgattcgacaatttggtgggacacttgaagtagacggtaaaaaaattatgttaactggaccgcaacaattaacaggtcaaaatgtggtagttcctggtgatatctcttctgcagctttctttttagttgcgggtttagtagtcccagatagcgagatacttctgaaaaatgttggcttaaatcaaacgcggacaggtattttagatgtgattaaaaacatgggcggttccgtcactattttaaatgaagatgaggccaatcattctggcgatttacttgtaaaaacgagtcaattaacagctacagagattggtggcgctattatcccacgtttaattgatgagttaccgattattgctttgttagctactcaggctactggcacgacaatcattcgagatgcagaagaattgaaagtcaaagaaaccaatcggattgatgcagtagcgaaagaattaacaattttaggcgccgacatcacgcctactgatgatggcttaattatacatggaccaacttctttacatggtggaagagttaccagttatggggatcatcgtatcgggatgatgttacaaattgctgcattacttgtaaaagaaggcactgttgaattagataaggctgaagcagtttcagtttcttatccagcattttttgacgacttagaacgtttaagttgttaa

SEQ ID NO: 26 - DNA, 1287 bp, Enterococcus faecalis
atgagggtgcaactacgtacaaatgtgaaacatttacaagggactctgatggttcctagcgacaaatcgatttcccatagaagtattatgtttggagcaatttcttctggaaaaacgacgattacaaattttctaagaggcgaagattgtttaagtaccttagcggcgtttcgttctttgggtgtgaacattgaagatgtcgggacgacaatcaccgttgaggggcaaggatttgcaggtttaaaaaaggcgaagaatacaattgatgttggaaattcagggacaacaattcgcctaatgctgggcattttagctggctgtccctttgaaacgcgcctagctggtgatgcgtctatttctaaacgaccgatgaatcgtgtgatgcttcctttaaaccaaatgggagcggaatgtcaaggggttcagcaaacggagtttccgccaatttctattcgcgggactcaaaatttgcaaccgattgactacacaatgcctgttgcgagtgctcaagtgaaatcggctattttattcgccgctttgcaagccgagggcacttctgtagtggttgagaaagaaaagacacgtgatcatacagaagagatgattcgacaatttggtgggacacttgaagtagacggtaaaaaaattatgttaactggaccgcaacaattaacaggtcaaaatgtggtagttcctggtgatatctcttctgcagctttctttttagttgcgggtttagtagtcccagatagcgagatacttctgaaaaatgttggcttaaatcaaacgcggacaggtattttagatgtgattaaaaacatgggtggttccgtcactattttaaatgaagatgaggccaatcactctggcgatttacttgtaaaaacgagtcaattgacagctacagagattggtggcgctattatcccacgtttaattgatgagttaccgattattgctttgttagctactcaggctactggcacgacaatcattcgagatgcagaagaattgaaagtcaaagaaaccaatcggattgatgcagtagcgaaagaattaacaattttaggcgccgacatcacgcctactgatgatggcttaattatacatgggccaacttctttacatggtggaagagttaccagttatggggatcatcgtatcgggatgatgttacaaattgctgcattacttgtaaaagaaggcactgttgaattagataaggctgaagcagtttcagtttcttatccagcattttttgacgacttagaacgtttaagttgttaa

SEQ ID NO: 27 - DNA, 870 bp, Enterococcus faecium
atgcgattattacaacaaatacatggattaagagggactgttaggataccagcagataaatcgatttctcatcgcagcatcatgtttggagcaattgctgagggaacgacgactatacaaaattttttgcgcgcagaagattgtctgagtactttacatgccttccaacaattaggcgtcgagatcgaagaagaggaagaggtgatcaagattcatggtcgcggtagccactcctttgtccaaccaactgcacccatcgacatgggaaactccggtacgacgagtcgtttattgatgggtattttggctggacagccttttacaacgactctggtcggtgatgcttcgttgtctaaacgtccaatggggcgagtgatggagcctttacgcgagatgggtgctgacttgcaaggaaatgaaagtgatcagtatctaccaatcactgtgacaggaacccgctctttatcaactatccgatacaatatgcctgtagctagtgcacaggtcaaatctgctttgctgtttgcggcactacaagcagaaggcacatccgtaatcgttgagaaagaacgttcccgtaaccatacggaagaaatgattcgtcaatttggtggaaggatcacagtggaagataaaacaatcatggtgacaggaccgcaaaaattaaccggtcagcagataactgttccaggtgatatttcatcagctgcattctttctagcagcaggacttcttgttccggaaagccagctgttgttaaaaaatgtcggggtcaatccaacaaggaccggtatcttagatgtgctagaggagatgggcgcacgattacccagacgaatcacaatgaacataaccaatcggctgatttaa

SEQ ID NO: 28 - DNA, 1065 bp, Thermotoga maritima
atgaaggtctttccgaagcccttcgctgagccaatagaacctctcttctgtggaaactccggaacaaccacgaggttgatgagtggagttcttgcttcatacgagatgttcacagtgctttatggggatccttctctctccagaaggccgatgagaagagtgatcgaacctctggagatgatgggagcgcgtttcatggcgaggcagaacaactaccttcccatggccatcaaaggaaatcacctttccggtatcagttacaaaacaccggtggcgagcgctcaagtgaagagcgctgttcttctggcggggctcagagccagcggacgaacaatcgttatcgaaccagcaaaaagcagagatcacacggaaaggatgctcaaaaacctcggtgttcccgtcgaggtggagggaacacgtgtggttctggagcctgctaccttcaggggtttcacgatgaaagtccctggtgatatctcgtcggctgctttcttcgtggttctcggcgccattcatcccaacgctcgaatcacagtaacggacgttggcctgaatcccacccgaacgggactcctcgaagttatgaaactcatgggagccaacctggagtgggagatcacggaagaaaatcttgaaccgataggaactgtgagggttgagacatctccaaacctgaaaggtgtggttgttcccgaacacctcgtacctctcatgatagatgaactgcctcttgtggcgcttctcggtgtttttgcggaaggagaaacggttgtgagaaacgcggaggagttgagaaagaaggaatccgacaggataagggttctggtggaaaacttcaaacggctcggtgtcgaaatagaagagttcaaagatggtttcaagatcgttggaaagcagagcataaaaggtggatcggtggatccagaaggcgaccacagaatggctatgctcttttccatagcagggctcgtgagtgaagagggggttgatgtgaaagatcacgaatgcgtggcggtgtctttcccgaacttttacgaactgctggagagagtggtgatatcatga

SEQ ID NO: 29 - DNA, 1296 bp, Aquifex aeolicus
atgaaaaaaatcgagaaaataaagagagttaaaggagaactcagagttccctccgacaagtccataacccacagggcttttatactgggggcactcgcaagcggtgaaactctagtaaggaaacctctaatctctggagacacactggccactttagaaatcctgaaagccatcagaacaaaagtaagggaaggaaaagaagaagtcttaattgagggaaggaattacacctttttagaacctcatgacgtactcgacgctaaaaactctgggactacggcgaggattatgagcggtgtactttctacacagcccttcttcagcgtccttacgggggacgaaagcctgaaaaacagaccgatgctgagagtggtggagcccttgagagagatgggggctaagatagatggaagggaggaggggaataaattaccgatagccataaggggaggaaacttaaagggaatttcctacttcaataaaaagtcctcagctcaagtaaagagtgccctcctgcttgcggggctgagagccgaaggtatgaccgaagttgtagaaccttacctttctcgtgatcacacagagagaatgttaaagctcttcggagcagaagtgataactattcctgaagaaaggggacacatagtaaaaataaaaggaggacaggaacttcagggaacggaagtttactgtcctgcggatccctcctctgcggcgtactttgcggcactcgctacgctcgctcctgaaggggagataagactaaaagaagttctcctgaatcctacccgtgacggattttacagaaaactcatagaaatgggaggggatatttcctttgaaaactacagggaactttccaacgaacctatggctgatcttgtagtaagacccgttgataacttaaaacccgtaaaggtttctcctgaagaagtacctactttaatagacgagattcccatccttgcggttcttatggcttttgcagacggagtatcggaggtaaagggagcgaaggaactcaggtacaaggaaagtgacaggataaaggctatagtcacaaacctaaggaagctcggagtacaggttgaggaatttgaggacggctttgcaattcacgggactaaagagataaagggaggagtgatagaaaccttcaaagatcacaggatagcgatggcttttgcagtgctcggattggtcgttgaagaggaagttataatagaccaccccgaatgcgttaccgtgtcttaccccgagttctgggaggatatcttaaaagtagtggagttctaa

SEQ ID NO: 30 - DNA, 1188 bp, Helicobacter pylori
atgggagaagattgtttaagctctttagaaatcgctcaaaatttaggggctaaagtggaaaataccgccaaaaattcttttaaaatcacacccccaacaactataaaagagcctaataagattttaaattgcaacaattctggcactagcatgcgtttatacagcgggcttttaagcgctcaaaaaggcctttttgttttaagcggggacaattccctaaacgcacgccccatgaaaagaatcattgagcctttaaaggcgtttggggcaaagattttagggagagaggataaccattttgcccccttagcgattgtagggggtcctttaaaagcttgcgattatgaaagccctatcgcttcagctcaagtcaaaagcgcttttattttaagcgccttacaagctcaaggcataagcgcctataaagaaagcgagcttagccgtaaccacacagaaatcatgcttaaaagtttgggggctaacattcaaaatcaagacggcgttttaaaaatttcacccctagaaaaacccctagaatcctttgactttaccatagccaatgatccgtctagcgcgttttttttagctctcgcttgcgcgattacgccaaaaagccgccttcttttaaaaaatgtcttgctcaaccccactcgcatagaagcttttgaggttttgaaaaaaatgggcgctcatatagaatatgttatccaatccaaagatttagaagttattggcgatatttacatagagcatgcccctttaaaagcgatcagtattgatcagaatatcgccagccttattgatgaaatccccgctttaagcatcgctatgctttttgcaaaaggcaaaagcatggtgagaaacgctaaagatttacgagccaaagaaagcgataggattaaagcggttgtttctaatttcaaagctttagggattgagtgcgaagaatttgaagacgggttttatatagagggattaggagatgcgagtcaattaaagcagcatttttctaagattaaaccccctattatcaagagtttcaatgatcacaggattgcgatgagtttcgctgttttaactttagcgttgcctttagaaattgataatttagaatgcgcgaacatttctttcccaacctttcagctttggctcaatctattcaaaaaaaggagtctcaatggaaattaa

SEQ ID NO: 31 - DNA, 1188 bp, Helicobacter pylori
atgggagaagattgtttaagctctttagaaatcgctcaaaatttaggggctaaagtggaaaataccgccaaaaattcttttaaaatcacacccccaacaactataaaggagcctaacaagattttaaattgcaacaattctggcacaaccatgcgtttatacagcgggcttttaagcgctcaaaaagggctttttgttttaagcggggacaattccttaaacgcacgccccatgaaaagaatcattgagcctttgaaggcttttggggcaaaaattttagggagagaggataaccatttcgcccccttagtgatcttagggagtccgttaaaagcttgccattatgaaagccctatcgcttcagctcaagtcaaaagcgcttttattttaagcgccttacaagctcaaggcgcaagcacttataaagaaagcgagcttagccgtaaccacacagaaatcatgcttaaaagtttgggagctgatattcacaatcaagacggcgttttaaaaatttcacccctagaaaaacccctagaagcctttgattttacgatagctaatgatccgtctagcgcgttttttttcgccctcgcttgcgcgattacgccaaaaagccgccttcttttaaaaaatgtcttgctcaaccccactcgcatagaagcttttgaagttttgaaaaaaatgggtgcttccatagagtatgcgattcagtccaaagatttagaaatgattggcgatatttatgtagagcatgcccctttaaaagcgatcaatattgatcaaaatatcgccagtcttattgatgaaatccccgctttaagtatcgctatgctttttgcaaaaggcaaaagcatggttaaaaacgctaaagatttacgagctaaagaaagcgacaggattaaagcggttgtttctaatttcaaagctttagggattgagtgcgaagagtttgaagatgggttttatgtagagggattagaagatataagcccattaaaacagcgcttttctaggattaagcccccccttatcaaaagcttcaatgaccacaggattgcgatgagttttgctgttttaactttagcgttgcctttagaaattgataatttagaatgcgcaaacatttctttcccgcaattcaaacacctactcaatcaattcaaaaaagggagtcttaatggaaattaa

SEQ ID NO: 32 - DNA, 1287 bp, Campylobacter jejuni
atgaaaatttacaaattgcaaacccctgtaaatgctatacttgaaaatatagcagcagataaaagcatatctcatcgttttgctatattttcgcttttaacacaagaagaaaataaggctcaaaattatctcttagctcaagatactttaaacactcttgaaattataaaaaatcttggagctaaaattgaacaaaaagattcttgcgtcaaaattataccccctaaagaaattttatctccaaattgtattttagactgtggaaattcaggaactgctatgcgtttgatgataggatttttagcaggaatttctggtttttttgttttaagtggagataagtatttaaacaatcgtcctatgagaaggataagcaaaccacttactcaaataggcgctagaatttatggaagaaatgaggcaaatttagctccactttgtatagaaggtcaaaaattaaaagcttttaattttaaaagcgaaatttcttcggctcaagttaaaacagctatgattttatctgcttttagggctgataatgtatgcacttttagtgaaatttctcttagtcgaaatcatagtgaaaacatgctaaaggctatgaaagctccaataagggttagtaatgatggcttaagtcttgaaataaatcctttaaaaaaacctttaaaagctcaaaatataatcattcctaatgatccttcttcggctttttattttgttttagcagctattattttacctaaatctcaaattattttaaaaaatattttgcttaatcctactcgtatagaggcgtataaaattttgcaaaaaatgggtgccaaacttgaaatgacaataactcaaaatgattttgaaactattggtgagatcagggtggagtctagcaagcttaatggcatagaagttaaagataatatcgcttggttaatagatgaagcgcctgctttggctatagcttttgctttggctaagggtaaatctagtttaataaatgctaaagaattacgcgttaaagaaagcgataggattgctgtgatggttgaaaatctaaagctttgtggtgttgaagctagagaacttgatgatggttttgaaatagaaggtggatgcgaactaaaatcttcaaaaattaaaagctatggagatcaccgtattgctatgagttttgctattttaggtttgctttgtggaatcgagattgatgatagtgattgtataaaaacttcttttccaaattttatagagattttatcaaatttaggagctaggattgattattga

SEQ ID NO: 33 - DNA, 1233 bp, Artificial Sequence
  Thermatoga EPSPS coding sequence designed with soybean codons
atgttgtccgtaccacctgacaagagcataactcacagagcacttatcttgtcagctctggcagagactgaatctactctctacaacctgttacgttgtctggacaccgagcgcacgcacgatattctggagaaactcggtacgaggttcgaaggagattgggaaaagatgaaggtgtttccgaagccctttgccgagcctatcgaaccactgttctgtggaaactcagggactactactaggttaatgtccggcgttcttgcgtcatacgaaatgtttacagtgctttacggtgatccgagtctatcaagacgacctatgaggagagttattgagcccttggagatgatgggcgctcggttcatggctcgccagaacaactacctacctatggctatcaaaggaaaccatctatctggaatttcctataagacgccagttgcgtctgctcaagtcaagtcggcagttctacttgccggtcttcgagcaagcgggagaactatcgtaatcgaaccagcgaaatcgcgtgaccatacggagaggatgctcaagaacctcggtgtgccagtagaggttgaaggaactcgtgtggttctcgaaccagctactttcagaggcttcacgatgaaggtgcctggtgatatatctagtgctgccttcttcgtggttctgggtgcaatccaccccaatgcgagaatcaccgtcacagacgttgggttaaaccctactaggaccggactcctggaagttatgaagctaatgggtgccaatttggagtgggaaatcaccgaggaaaaccttgagcctatcggaacagttagagtggaaacatcgcctaacctgaaaggagtggtcgttcctgagcaccttgttccacttatgattgatgagttgccgctcgtcgctctcctgggtgtcttcgcggaaggagagacagttgtcagaaacgcagaagagctaaggaagaaggaatcagatcggatcagagtgctcgttgagaatttcaagcgattgggtgtggaaattgaagagttcaaagacggcttcaagatcgtcggcaaacagtcgatcaaaggaggttcagttgatccggaaggagaccacagaatggctatgctgtttagtatagccggacttgtgtccgaggaaggtgtggacgtaaaagatcacgaatgtgtcgctgtgagctttccaaacttctacgagttgctagaaagagtcgttatctcttaa

SEQ ID NO: 34 - DNA, 1332 bp, Artificial Sequence
  Caulobacter EPSPS coding sequence designed with Arabadopsis codons
atgtccctagcgggtctgaagtctgctcccggtggagcactaagagggatcgtgcgcgctccaggcgataagtcaattagtcaccggtccatgattctaggtgctctggcaaccggtacaactaccgttgaagggctattggaaggcgatgacgtacttgcgactgccagagctatgcaagccttcggtgcacggatagagcgagagggtgtcggacgctggcgtatcgaaggcaaaggtggctttgaggaaccggttgacgtgattgattgtgggaacgctggcaccggtgtacgactcattatgggtgcagccgcagggttcgcaatgtgtgccaccttcactggagatcaatctctaagaggacgaccaatgggcagagtgttagatcctctcgccaggatgggtgcgacatggctaggacgggataaaggacggctcccacttacactcaagggtggaaatcttcgtggactgaactacacacttccgatggcctcggctcaagttaagtcagcagtattgcttgccggactccacgcggaaggtggagttgaagtcatcgagcctgaagctacgagagaccacacagaacggatgcttagggctttcggagcagaagtaatcgttgaggaccgtaaggctggtgataagacattccgccatgtgaggctgcctgagggacagaaactcacgggcacgcacgttgcggtcccaggcgatccgtcatctgccgcgttcccactggttgctgcgctgatagtgcctggttcggaagtaactgtggaaggtgtcatgctcaacgaacttcgaacagggttgttcactacgttacaggagatgggagctgatctggtcatctccaacgttcgtgtagcctcaggcgaggaagtaggagacatcactgcgcgatattcgcagctaaaaggtgttgtagtgccacctgagcgtgctccgtctatgatcgacgaatacccgatactcgccgtcgcagccgcgttcgcttctggcgaaaccgtgatgagaggtgtaggagagatgcgggtcaaagagagcgaccgtatcagcttgacggccaacggtcttaaggcttgcggagttcaagtagtggaggaacctgagggctttattgttacgggtactgggcaaccaccgaaaggaggtgccaccgtggtcacgcatggagatcaccgcattgctatgagtcacctaatcttggggatggcagctcaagcagaggtcgcggtggatgaacccggtatgatagccactagcttcccaggattcgcggatctgatgagagggttaggagcaacgttggcagaggcttga

SEQ ID NO: 35 - DNA, 1341 bp, Artificial Sequence
  Xanthomonas EPSPS coding sequence designed with Arabidopsis codons
atgagttccgttagtaccgcttgcatgagtaactccactcagcactggatcgcgcagcgcgggactgcccttcaaggctcacttactatccctggtgataagtccgttagtcatagagctgttatgtttgctgcacttgctgacgggattagcaagatcgacggattcctagaaggtgaggataccaggagtaccgctgccatcttcgcacaacttggcgtgcgtattgaaacaccttctgcgtcgcaacggatcgtccacggagtcggagttgacggccttcaaccacctcagggtcctcttgactgcggaaacgccggcactggaatgagactgctggctggtgtacttgcagcccagcggttcgactcagtcctcgttggagacgcttcgctctcgaaacgtcccatgagacgagtgaccggcccgcttgctcagatgggtgctagaatcgagacggagtccgacggtacacctccactcagggtccacggtgggcaagcacttcaaggcatcactttcgcgtctccagtcgcttccgctcaagtcaaatctgcagtcctgcttgctggactctacgctactggagagacatctgtgtccgaaccgcatcccactagagattacaccgagagaatgctatcagccttcggagtagagatcgcgtttagtccaggacaagcgagattgcgtggaggccagcgcttgcgtgctacagatattgctgtgcctgctgacttctcctcagcagcattcttcatcgtcgctgcctctatcattcctggttctggagttaccctcagggctgttggactcaatcctagacgcaccggtctcttggcagcgctcaggctaatgggcgccgatattgttgaggacaatcacgccgagcacggaggtgagccagtggccgatctgcgtgttcgatacgcacccttgcgtggtgctcagattccagaagccctggttccggatatgatcgacgagtttccggccttgttcgtcgctgccgctgcggcacgaggtgatacggttgtgtctggtgctgcagaactaagggtcaaggaatctgacaggcttgcagcgatggctactgggctccgagcattagggattgttgtcgatgagacacctgatggagcaacaattcacggcggtacactcggttccggtgtaatcgaatctcatggagatcataggatagctatggcattcgctatcgctggtcagctatcaaccggtacggttcaagtcaacgatgtggctaacgtagccacctccttcccaggattcgactcgttagctcagggtgcgggattcgggcttagtgcacgtccctga

SEQ ID NO: 36 - DNA, 1331 bp, Artificial Sequence
  Caulobacter EPSPS coding seqeunce designed with monocot codons
atgagcctagccggtcttaagtccgctcctggcggtgcccttcgcgggatcgtgagggctcccggtgacaagagcatctcacataggtcgatgattctaggcgcgttagcaaccgggactacaactgttgagggcctccttgagggtgacgacgtcctcgccaccgctagggcgatgcaagccttcggtgcccggatcgaacgcgagggagtgggcagatggcggattgagggcaagggtggctttgaggaacccgtagacgtgattgattgcggaaacgcgggcactggtgtgcgtttgattatgggcgctgccgctggcttcgcgatgtgtgccacctttaccggtgaccagtcactgcgcggtaggccgatgggacgggttctcgaccctctcgccagaatgggcgctacctggctgggaagggataagggtaggttgccactcacgctgaaaggtggcaatctgcgcggactcaactacacgctgccgatggcgtccgctcaagttaagtctgccgttctccttgctggcctgcacgctgaaggtggcgtggaagtcatcgagcctgaggcgacgcgcgatcacaccgagcgcatgttgcgtgcattcggtgccgaggtcatcgtggaggataggaaggctggcgacaagacgttcaggcacgtccgtctgccagagggccagaagctcaccggcactcacgttgctgtacccggtgacccgtcctctgccgcgttcccgctcgtggctgcactgatcgtcccaggctctgaggtcaccgtggagggcgtgatgctcaacgaacttagaacaggactgtttaccacgctccaagaaatgggagcggaccttgtgatctccaacgttcgtgtcgcctctggagaggaagtgggcgatattaccgctcggtactcgcagctcaagggcgtcgtggtcccacctgagagagcaccaagtatgatcgacgaatatccgatcctggcggtcgcggcagcgttcgccagcggtgagaccgttatgcgcggcgtcggtgagatgcgcgtgaaggagtcggatcgaatcagtctcactgcaaacgggctgaaagcctgcggcgttcaagtggttgaggaacccgagggattcatcgttaccgggacagggcagcctcccaagggaggagccactgtcgttacccacggagatcaccggattgctatgtcacatcttattcttgggatggccgctcaggctgaggtcgcagtcgatgagcctgggatgatagccactagcttccctgggttcgcagacctgatgcgcgggttaggcgcgacactcgccgaggcttg

SEQ ID NO: 37 - DNA, 1316 bp, Artificial Sequence
  Xanthomonas EPSPS coding sequence designed with monocot codons
atgagcaactccacccagcactggatcgcccagcgcggcaccgccctccagggtagcctgacgatccctggtgacaagtcagtgagccatagggccgtgatgttcgctgccctagccgacgggattagcaagattgacggcttcctagagggcgaggatacgcgctcgactgctgcgatcttcgcacagcttggcgttaggatcgagacacccagcgcgtcgcagaggatcgtccacggcgttggagtggacggcttgcaacctcctcagggacccttggattgcggcaacgcaggcactgggatgaggctgctcgcaggcgtcctggcagctcagcgtttcgactctgtcctggtgggtgacgcctctttgtccaagcgtccgatgaggagagtcaccggtccgcttgcccaaatgggtgcgaggatcgagaccgagtccgacggtacgcctccactccgggtgcacggaggccaggcgctgcaagggatcacctttgcctctcccgtcgcttccgcccaagtcaagagtgctgtcctgctcgctggcctttacgccacaggcgaaacctcggttagcgagcctcacccgacccgcgactacactgagcgaatgctgtcggcgttcggcgtggagattgcgtttagcccagggcaagcgagacttcgcggtggtcagcggcttcgcgcaactgacatcgccgttccagccgacttcagttctgctgcattctttatcgtcgctgctagcatcattcccggatctggcgtcacgctccgtgctgtcggactgaacccacggaggactggcctccttgctgccctccgattgatgggtgcggacatcgtggaggacaatcacgctgagcacggcggtgagccggttgccgacctgcgcgttcgctatgcaccgctgcgaggtgcgcagattccggaagcgctggttcccgacatgatcgacgagttccctgccctctttgtcgcagccgctgcggcacgcggcgatactgtggtatccggagctgcggagctgagggtgaaagaatccgatagactcgcggctatggcaactgggctccgcgctctagggatagtggttgacgagactcccgatggtgccacgatccacggcggaacattagggagtggtgtgatagaatcacatggcgatcaccgcattgctatggctttcgctatcgccgggcagctttcaacagggacagtgcaagtcaacgatgtggccaatgtggcgacgtccttcccagggttcgatagtcttgcccagggagccgggttcggattaagtgcccgtccttg

SEQ ID NO: 38 - DNA, 210 bp, Artificial Sequence
  modified polynucleotide sequence encodin a wheat GBSS CTP
atggcggcactggtgacctcccagctcgcgacaagcggcaccgtcctgtcggtgacggaccgcttccggcgtcccggcttccagggactgaggccacggaacccagccgatgccgctctcgggatgaggacggtgggcgcgtccgcggctcccaagcagagcaggaagccacaccgtttcgaccgccggtgcttgagcatggtcgtctgc

SEQ ID NO: 39 - DNA, 1578 bp, Artificial Sequence
  polynucleotide encoding a wheat GBSS CTP fused to CP4 EPSPS coding sequence
atggcggcactggtgacctcccagctcgcgacaagcggcaccgtcctgtcggtgacggaccgcttccggcgtcccggcttccagggactgaggccacggaacccagccgatgccgctctcgggatgaggacggtgggcgcgtccgcggctcccaagcagagcaggaagccacaccgtttcgaccgccggtgcttgagcatggtcgtctgcatgctacacggtgcaagcagccggccggcaaccgctcgcaaatcttccggcctttcgggaacggtcaggattccgggcgataagtccatatcccaccggtcgttcatgttcggcggtcttgccagcggtgagacgcgcatcacgggcctgcttgaaggtgaggacgtgatcaataccgggaaggccatgcaggctatgggagcgcgtatccgcaaggaaggtgacacatggatcattgacggcgttgggaatggcggtctgctcgcccctgaggcccctctcgacttcggcaatgcggcgacgggctgcaggctcactatgggactggtcggggtgtacgacttcgatagcacgttcatcggagacgcctcgctcacaaagcgcccaatgggccgcgttctgaacccgttgcgcgagatgggcgtacaggtcaaatccgaggatggtgaccgtttgcccgttacgctgcgcgggccgaagacgcctaccccgattacctaccgcgtgccaatggcatccgcccaggtcaagtcagccgtgctcctcgccggactgaacactccgggcatcaccacggtgatcgagcccatcatgaccagggatcataccgaaaagatgcttcaggggtttggcgccaacctgacggtcgagacggacgctgacggcgtcaggaccatccgccttgagggcaggggtaaactgactggccaagtcatcgatgttccgggagacccgtcgtccacggccttcccgttggttgcggcgctgctcgtgccggggagtgacgtgaccatcctgaacgtcctcatgaacccgaccaggaccggcctgatcctcacgcttcaggagatgggagccgacatcgaggtgatcaacccgcgcctggcaggcggtgaagacgttgcggatctgcgcgtgcgctcctctaccctgaagggcgtgacggtcccggaagatcgcgcgccgtccatgatagacgagtatcctattctggccgtcgccgctgcgttcgccgaaggggccacggtcatgaacggtcttgaggaactccgcgtgaaggaatcggatcgcctgtcggcggtggccaatggcctgaagctcaacggtgttgactgcgacgagggtgagacctcactcgtggtccgtggccggcctgatggcaagggcctcggcaacgccagtggagcggccgtcgccacgcacctcgatcatcgcatcgcgatgtccttcttggtgatgggtctcgtctcagagaacccggtgaccgtcgatgacgccacgatgatagcgacgagcttcccagagttcatggatctgatggcgggcctcggggccaagatcgaactgtctgacacgaaggccgcttga

SEQ ID NO: 40 - DNA, 1527 bp, Artificial Sequence
  polynucleotide encoding a wheat GBSS CTP fused with an artificial sequence encoding a Xanthomonas EPSPS
atggcagcgctggtgactagccagctcgccacaagcggcaccgtcctgtcggtgacggaccgcttccggcgtcccggcttccagggactgaggccacggaacccagcggacgctgccctcgggatgaggacggtgggcgcgtccgctgcgcccaagcagagtaggaagccacatcgcttcgaccgtcggtgcttgagtatggtcgtctgcatgagcaactccacccagcactggatcgcccagcgcggcaccgccctccagggtagcctgacgatccctggtgacaagtcagtgagccatagggccgtgatgttcgctgccctagccgacgggattagcaagattgacggcttcctagagggcgaggatacgcgctcgactgctgcgatcttcgcacagcttggcgttaggatcgagacacccagcgcgtcgcagaggatcgtccacggcgttggagtggacggcttgcaacctcctcagggacccttggattgcggcaacgcaggcactgggatgaggctgctcgcaggcgtcctggcagctcagcgtttcgactctgtcctggtgggtgacgcctctttgtccaagcgtccgatgaggagagtcaccggtccgcttgcccaaatgggtgcgaggatcgagaccgagtccgacggtacgcctccactccgggtgcacggaggccaggcgctgcaagggatcacctttgcctctcccgtcgcttccgcccaagtcaagagtgctgtcctgctcgctggcctttacgccacaggcgaaacctcggttagcgagcctcacccgacccgcgactacactgagcgaatgctgtcggcgttcggcgtggagattgcgtttagcccagggcaagcgagacttcgcggtggtcagcggcttcgcgcaactgacatcgccgttccagccgacttcagttctgctgcattctttatcgtcgctgctagcatcattcccggatctggcgtcacgctccgtgctgtcggactgaacccacggaggactggcctccttgctgccctccgattgatgggtgcggacatcgtggaggacaatcacgctgagcacggcggtgagccggttgccgacctgcgcgttcgctatgcaccgctgcgaggtgcgcagattccggaagcgctggttcccgacatgatcgacgagttccctgccctctttgtcgcagccgctgcggcacgcggcgatactgtggtatccggagctgcggagctgagggtgaaagaatccgatagactcgcggctatggcaactgggctccgcgctctagggatagtggttgacgagactcccgatggtgccacgatccacggcggaacattagggagtggtgtgatagaatcacatggcgatcaccgcattgctatggctttcgctatcgccgggcagctttcaacagggacagtgcaagtcaacgatgtggccaatgtggcgacgtccttcccagggttcgatagtcttgcccagggagccgggttcggattaagtgcccgtccttga

SEQ ID NO: 41 - DNA, 1542 bp, Artificial Sequence
  polynucleotide encoding a wheat GBSS CTP fused to a Caulobacter EPSPS coding sequence
atggcagcgctggtgactagccagctcgccacaagcggcaccgtcctgtcggtgacggaccgcttccggcgtcccggcttccagggactgaggccacggaacccagcggacgctgccctcgggatgaggacggtgggcgcgtccgctgcgcccaagcagagtaggaagccacatcgcttcgaccgtcggtgcttgagtatggtcgtctgcatgagcctagccggtcttaagtccgctcctggcggtgcccttcgcgggatcgtgagggctcccggtgacaagagcatctcacataggtcgatgattctaggcgcgttagcaaccgggactacaactgttgagggcctccttgagggtgacgacgtcctcgccaccgctagggcgatgcaagccttcggtgcccggatcgaacgcgagggagtgggcagatggcggattgagggcaagggtggctttgaggaacccgtagacgtgattgattgcggaaacgcgggcactggtgtgcgtttgattatgggcgctgccgctggcttcgcgatgtgtgccacctttaccggtgaccagtcactgcgcggtaggccgatgggacgggttctcgaccctctcgccagaatgggcgctacctggctgggaagggataagggtaggttgccactcacgctgaaaggtggcaatctgcgcggactcaactacacgctgccgatggcgtccgctcaagttaagtctgccgttctccttgctggcctgcacgctgaaggtggcgtggaagtcatcgagcctgaggcgacgcgcgatcacaccgagcgcatgttgcgtgcattcggtgccgaggtcatcgtggaggataggaaggctggcgacaagacgttcaggcacgtccgtctgccagagggccagaagctcaccggcactcacgttgctgtacccggtgacccgtcctctgccgcgttcccgctcgtggctgcactgatcgtcccaggctctgaggtcaccgtggagggcgtgatgctcaacgaacttagaacaggactgtttaccacgctccaagaaatgggagcggaccttgtgatctccaacgttcgtgtcgcctctggagaggaagtgggcgatattaccgctcggtactcgcagctcaagggcgtcgtggtcccacctgagagagcaccaagtatgatcgacgaatatccgatcctggcggtcgcggcagcgttcgccagcggtgagaccgttatgcgcggcgtcggtgagatgcgcgtgaaggagtcggatcgaatcagtctcactgcaaacgggctgaaagcctgcggcgttcaagtggttgaggaacccgagggattcatcgttaccgggacagggcagcctcccaagggaggagccactgtcgttacccacggagatcaccggattgctatgtcacatcttattcttgggatggccgctcaggctgaggtcgcagtcgatgagcctgggatgatagccactagcttccctgggttcgcagacctgatgcgcgggttaggcgcgacactcgccgaggcttga

SEQ ID NO: 42 - DNA, 36 bp, Thermotoga maritima
ctagtccatatgctgagcgttcctccggacaaatcc

SEQ ID NO: 43 - DNA, 37 bp, Thermotoga maritima
ctgatctgatcatcatgatatcaccactctctccagc

SEQ ID NO: 44 - DNA, 34 bp, Caulobacter sp.
caagcatatgtcgctggctggattgaagagcgct

SEQ ID NO: 45 - DNA, 40 bp, Caulobacter sp.
ggggagatctctcgagttatcaggcctccgccagcgtcgc

SEQ ID NO: 46 - DNA, 29 bp, Xanthomonas campestris
ccacatatgagcaacagcacgcaacactg

SEQ ID NO: 47 - DNA, 29 bp, Xanthomonas campestris
caactcgagtcacggacgcgcgctgagcc

SEQ ID NO: 48 - DNA, 28 bp, Campylobacter jejuni
ctagtccatatgaaaatttacaaattgc

SEQ ID NO: 49 - DNA, 30 bp, Campylobacter jejuni
ctgatcggatcctcaataatcaatcctagc

SEQ ID NO: 50 - DNA, 33 bp, Helicobacter pylori
ctagtccatatgatagagcttgacattaacgcc

SEQ ID NO: 51 - DNA, 40 bp, Helicobacter pylori
ctgatcggatccttaatttccattgagactcctttttttg